(19) United States
(12) Patent Application Publication
(10) Pub. No.: US 2025/0366701 A1
(43) Pub. Date: Dec. 4, 2025

(54) MEDICAL SUPPORT DEVICE, ENDOSCOPE, MEDICAL SUPPORT METHOD, AND PROGRAM

(71) Applicant: FUJIFILM Corporation, Tokyo (JP)

(72) Inventor: Misaki GOTO, Kanagawa (JP)

(73) Assignee: FUJIFILM Corporation, Tokyo (JP)

(21) Appl. No.: 19/303,309

(22) Filed: Aug. 18, 2025

Related U.S. Application Data
(63) Continuation of application No. PCT/JP2024/003504, filed on Feb. 2, 2024.

(30) Foreign Application Priority Data
Feb. 21, 2023 (JP) 2023-025534

(51) Int. Cl. A61B 1/00; G06T 7/00; G06T 7/60

(52) U.S. Cl. CPC A61B 1/000094; A61B 1/000096; G06T 7/0012; G06T 7/60; G06T 2207/10068

(57) ABSTRACT

A medical support device includes a processor. The processor is configured to recognize, using a medical image, an observation target region appearing in the medical image, measure a size corresponding to a characteristic of the observation target region, based on the medical image, and output the size.

### CROSS-REFERENCE TO RELATED APPLICATIONS

This application is a continuation application of International Application No. PCT/JP2024/003504 filed Feb. 2, 2024, the disclosure of which is incorporated herein by reference in its entirety. Further, this application claims priority from Japanese Patent Application No. 2023-025534, filed Feb. 21, 2023, the disclosure of which is incorporated herein by reference in its entirety.

### BACKGROUND

### 1. Technical Field

The technology of the present disclosure relates to a medical support device, an endoscope, a medical support method, and a program.

### 2. Related Art

JP2008-061704A discloses an image display apparatus that displays an image group obtained by imaging the inside of a subject along a time series. The image display apparatus described in JP2008-061704A includes an image detection means, a mark display means, and a display control means.

The image detection means detects lesion images included in the image group. The mark display means displays lesion marks along a time bar indicating an overall time position of the image group, the lesion marks indicating time positions of the lesion images on the time bar. The display control means calculates the number of images per unit pixel of the time bar, based on the number of pixels in a time axis direction that defines the time bar and based on the number of images in the image group. Further, the display control means counts the number of lesion images for each consecutive image group in the image group in which a number of images equal to the number of images per unit pixel are consecutive. Then, the display control means performs control to display, for each consecutive image group including one or more lesion images, a lesion mark having a display format corresponding to the counting result of the number of lesion images.

WO2020/165978A discloses an image recording apparatus having an acquisition unit that acquires time-series images of endoscopy, a lesion appearance identification unit that identifies an appearance of a lesion in the acquired time-series images, and a recording unit that starts recording of the time-series images from a time point when the appearance of the lesion is identified by the lesion appearance identification unit.

In the image recording apparatus described in WO2020/165978A, the lesion appearance identification unit has a lesion detection unit that detects a lesion based on the acquired time-series images. The lesion appearance identification unit further has a lesion information calculation unit that calculates information related to the lesion, based on the lesion detected by the lesion detection unit. The lesion information calculation unit calculates information on a size of the lesion detected by the lesion detection unit.

### SUMMARY

An embodiment according to the technology of the present disclosure provides a medical support device, an endoscope, a medical support method, and a program that enable a user or the like to accurately grasp the size of an observation target region appearing in a medical image.

A first aspect according to the technology of the present disclosure is a medical support device including a processor, the processor being configured to recognize, using a medical image, an observation target region appearing in the medical image, measure a size corresponding to a characteristic of the observation target region, based on the medical image, and output the size.

A second aspect according to the technology of the present disclosure is the medical support device according to the first aspect, in which the characteristic includes a shape of the observation target region, a category of the observation target region, a type of the observation target region, clarity of a contour of the observation target region, and/or an overlap between the observation target region and a peripheral region.

A third aspect according to the technology of the present disclosure is the medical support device according to the first aspect or the second aspect, in which the processor is configured to recognize the characteristic, based on the medical image.

A fourth aspect according to the technology of the present disclosure is the medical support device according to any one of the first to third aspects, in which the size is a long side, a short side, a radius, and/or a diameter of the observation target region.

A fifth aspect according to the technology of the present disclosure is the medical support device according to any one of the first to fourth aspects, in which the observation target region is recognized by a method using AI, and the size is measured based on a probability map obtained from the AI.

A sixth aspect according to the technology of the present disclosure is the medical support device according to the fifth aspect, in which the size is measured based on a closed region obtained by dividing the probability map according to a threshold value.

A seventh aspect according to the technology of the present disclosure is the medical support device according to the fifth aspect or the sixth aspect, in which the size is measured based on a plurality of segment regions obtained by dividing the probability map according to a plurality of threshold values.

An eighth aspect according to the technology of the present disclosure is the medical support device according to the seventh aspect, in which the size has a range, and the range is identified based on the plurality of segment regions.

A ninth aspect according to the technology of the present disclosure is the medical support device according to the eighth aspect, in which a lower limit value of the range is measured based on a first segment region that is smallest among the plurality of segment regions, and an upper limit value of the range is measured based on a second segment region that is an outer region with respect to the first segment region among the plurality of segment regions.

A tenth aspect according to the technology of the present disclosure is the medical support device according to any one of the first to ninth aspects, in which the processor is configured to measure a plurality of first sizes of the observation target region, based on the medical image, and the size is a representative value of the plurality of first sizes.

An eleventh aspect according to the technology of the present disclosure is the medical support device according to the tenth aspect, in which the representative value includes a maximum value, a minimum value, a mean value, a median value, and/or a variance value.

A twelfth aspect according to the technology of the present disclosure is the medical support device according to any one of the first to eleventh aspects, in which the characteristic includes an overlap between the observation target region and a peripheral region, and the size is a size of the observation target region in a case where the overlap is included and/or a size of the observation target region in a case where the overlap is not included.

A thirteenth aspect according to the technology of the present disclosure is the medical support device according to any one of the first to twelfth aspects, in which the size is output by displaying the size on a screen.

A fourteenth aspect according to the technology of the present disclosure is the medical support device according to any one of the first to thirteenth aspects, in which the medical image is an endoscopic image obtained by imaging with an endoscope.

A fifteenth aspect according to the technology of the present disclosure is the medical support device according to any one of the first to fourteenth aspects, in which the observation target region is a lesion.

A sixteenth aspect according to the technology of the present disclosure is an endoscope including the medical support device according to any one of the first to fifteenth aspects, and a module to be inserted into a body including the observation target region to acquire the medical image by imaging the observation target region.

A seventeenth aspect according to the technology of the present disclosure is a medical support method including recognizing, using a medical image, an observation target region appearing in the medical image; measuring a size corresponding to a characteristic of the observation target region, based on the medical image; and outputting the size.

An eighteenth aspect according to the technology of the present disclosure is a program for causing a computer to execute a medical support process, the medical support process including recognizing, using a medical image, an observation target region appearing in the medical image; measuring a size corresponding to a characteristic of the observation target region, based on the medical image; and outputting the size.

### BRIEF DESCRIPTION OF THE DRAWINGS

Exemplary embodiments according to the technique of the present disclosure will be described in detail based on the following figures, wherein:

FIG. 1 is a conceptual diagram illustrating an example of an aspect in which an endoscope system is used;

FIG. 2 is a conceptual diagram illustrating an example overall configuration of an endoscope system;

FIG. 3 is a block diagram illustrating an example hardware configuration of an electric system of the endoscope system;

FIG. 4 is a block diagram illustrating an example of functions of main components, according to a first embodiment, of a processor included in the endoscope, and an example of information according to the first embodiment, which is stored in an NVM;

FIG. 5 is a conceptual diagram illustrating an example of the content of a process performed by a recognition unit and a control unit according to the first embodiment;

FIG. 6 is a conceptual diagram illustrating an example of the content of a process performed by a measurement unit to measure a minimum size;

FIG. 7 is a conceptual diagram illustrating an example of the content of a process performed by the measurement unit to measure a maximum size;

FIG. 8 is a conceptual diagram illustrating an example of an aspect in which an endoscopic image is displayed in a first display region and a size is displayed on a map in a second display region;

FIG. 9 is a flowchart illustrating an example of the flow of a medical support process;

FIG. 10 is a conceptual diagram illustrating an example of an aspect in which a size is displayed in the endoscopic image;

FIG. 11 is a conceptual diagram illustrating an example of an aspect in which sizes in a plurality of directions are displayed on a probability map;

FIG. 12 is a block diagram illustrating an example of functions of main components, according to a second embodiment, of the processor included in the endoscope, and an example of information according to the second embodiment, which is stored in the NVM;

FIG. 13 is a conceptual diagram illustrating an example of the content of a process performed by the recognition unit and the control unit according to the second embodiment;

FIG. 14 is a conceptual diagram illustrating an example of the content of a process performed by a generation unit;

FIG. 15 is a conceptual diagram illustrating an example of the content of a process performed by the recognition unit according to the second embodiment;

FIG. 16 is a conceptual diagram illustrating an example of the content of a process performed by the measurement unit according to the second embodiment;

FIG. 17 is a conceptual diagram illustrating an example of an aspect in which the control unit according to the second embodiment displays a visible size in the second display region;

FIG. 18 is a conceptual diagram illustrating an example of an aspect in which the control unit according to the second embodiment displays a predicted size in the second display region;

FIG. 19 is a conceptual diagram illustrating an example of an aspect in which when a lesion appearing in the endoscopic image is pedunculated, the control unit displays the visible size of the lesion in the second display region; and

FIG. 20 is a conceptual diagram illustrating examples of a size output destination.

### DETAILED DESCRIPTION

An example of an embodiment of a medical support device, an endoscope, a medical support method, and a program according to the technology of the present disclosure will be described hereinafter with reference to the accompanying drawings.

First, terms used in the following description will be described.

CPU is an abbreviation for “Central Processing Unit”. GPU is an abbreviation for “Graphics Processing Unit”. RAM is an abbreviation for “Random Access Memory”. NVM is an abbreviation for “Non-volatile memory”. EEPROM is an abbreviation for “Electrically Erasable Programmable Read-Only Memory”. ASIC is an abbreviation for “Application Specific Integrated Circuit”. PLD is an abbreviation for “Programmable Logic Device”. FPGA is an abbreviation for “Field-Programmable Gate Array”. SoC is an abbreviation for “System-on-a-chip”. SSD is an abbreviation for “Solid State Drive”. USB is an abbreviation for “Universal Serial Bus”. HDD is an abbreviation for “Hard Disk Drive”. EL is an abbreviation for “Electro-Luminescence”. CMOS is an abbreviation for “Complementary Metal Oxide Semiconductor”. CCD is an abbreviation for “Charge Coupled Device”. AI is an abbreviation for “Artificial Intelligence”. BLI is an abbreviation for “Blue Light Imaging”. LCI is an abbreviation for “Linked Color Imaging”. I/F is an abbreviation for “Interface”. SSL is an abbreviation for “Sessile Serrated Lesion”. GANs is an abbreviation for “Generative Adversarial Networks”. VAE is an abbreviation for “Variational Autoencoder”.

### First Embodiment

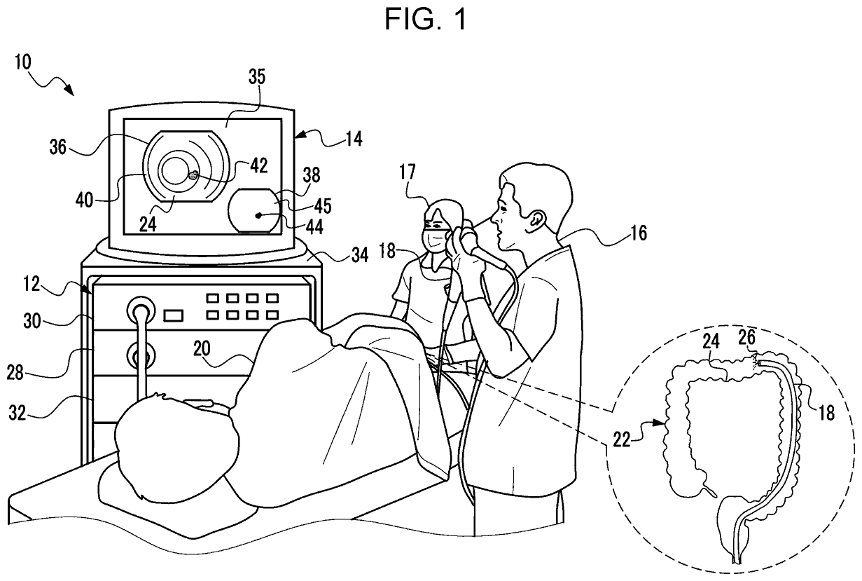

As an example, as illustrated in FIG. 1, an endoscope system 10 includes an endoscope 12 and a display device 14. The endoscope 12 is used by a doctor 16 in an endoscopic examination. The endoscopic examination is assisted by staff such as a nurse 17. In the first embodiment, the endoscope 12 is an example of an “endoscope” according to the technology of the present disclosure.

The endoscope 12 is connected to a communication device (not illustrated) in a communicable manner, and information obtained by the endoscope 12 is transmitted to the communication device. An example of the communication device is a server and/or a client terminal (for example, a personal computer and/or a tablet terminal) that manages various kinds of information such as electronic medical records. The communication device receives the information transmitted from the endoscope 12 and executes a process using the received information (for example, a process of storing the information in an electronic medical record or the like).

The endoscope 12 includes an endoscope main body 18. The endoscope 12 is an apparatus for performing medical care for a large intestine 22 included in the body of a subject 20 (for example, a patient) using the endoscope main body 18. In the first embodiment, the large intestine 22 is a target to be observed by the doctor 16.

The endoscope main body 18 is inserted into the large intestine 22 of the subject 20. The endoscope 12 causes the endoscope main body 18 inserted into the large intestine 22 of the subject 20 to perform imaging of the inside of the large intestine 22 in the body of the subject 20, and performs various medical treatments on the large intestine 22 as necessary.

The endoscope 12 performs imaging of the inside of the large intestine 22 of the subject 20 to acquire an image indicating the state of the inside of the body, and outputs the acquired image. In the first embodiment, the endoscope 12 is an endoscope having an optical imaging function of capturing an image of reflected light obtained by irradiating the inside of the large intestine 22 with light 26 and reflecting the light 26 from an intestinal wall 24 of the large intestine 22.

While the endoscopic examination of the large intestine 22 is illustrated here, this is merely an example, and the technology of the present disclosure is also applicable to an endoscopic examination of a luminal organ such as the esophagus, the stomach, the duodenum, or the trachea.

The endoscope 12 includes a control device 28, a light source device 30, and a medical support device 32. The control device 28, the light source device 30, and the medical support device 32 are installed in a cart 34. The cart 34 is provided with a plurality of shelves along the vertical direction, and the medical support device 32, the control device 28, and the light source device 30 are installed on the shelves from bottom to top. The display device 14 is installed on top of the cart 34.

The control device 28 controls the entire endoscope 12. The medical support device 32 performs various kinds of image processing on an image obtained by imaging of the intestinal wall 24 using the endoscope main body 18, under the control of the control device 28.

The display device 14 displays various kinds of information including images. An example of the display device 14 is a liquid crystal display or an EL display. A tablet terminal with a display may be used instead of or together with the display device 14.

The display device 14 displays a screen 35. In the first embodiment, the screen 35 is an example of a “screen” according to the technology of the present disclosure. The screen 35 includes a plurality of display regions. The plurality of display regions are arranged side by side on the screen 35. In the example illustrated in FIG. 1, a first display region 36 and a second display region 38 are illustrated as an example of the plurality of display regions. The size of the first display region 36 is larger than the size of the second display region 38. The first display region 36 is used as a main display region, and the second display region 38 is used as a sub-display region.

The first display region 36 displays an endoscopic image 40. The endoscopic image 40 is an image acquired by imaging of the intestinal wall 24 in the large intestine 22 of the subject 20 using the endoscope main body 18. In the example illustrated in FIG. 1, an image in which the intestinal wall 24 appears is illustrated as an example of the endoscopic image 40. In the first embodiment, the endoscopic image 40 is an example of a “medical image” and an “endoscopic image” according to the technology of the present disclosure.

The intestinal wall 24 appearing in the endoscopic image 40 includes a lesion 42 (for example, in the example illustrated in FIG. 1, one lesion 42) as a region of interest (that is, an observation target region) to be gazed at by the doctor 16, and the doctor 16 can visually recognize the state of the intestinal wall 24 including the lesion 42 through the endoscopic image 40. In the first embodiment, the lesion 42 is an example of an “observation target region” and a “lesion” according to the technology of the present disclosure.

There are various types of lesions 42, and the types of lesions 42 include, for example, a neoplastic polyp and a non-neoplastic polyp. Examples of the type of neoplastic polyp include adenomatous polyps (for example, SSL). Examples of the type of non-neoplastic polyp include hamartoma polyps, hyperplastic polyps, and inflammatory polyps. The types illustrated here are types considered in advance to be types of lesions 42 when an endoscopic examination is performed on the large intestine 22, and the type of lesion differs depending on the organ on which the endoscopic examination is performed.

While the first embodiment provides an example embodiment in which one lesion 42 appears in the endoscopic image 40 for convenience of description, the technology of the present disclosure is not limited to this, and the technology of the present disclosure is also applicable in a case where a plurality of lesions 42 appear in the endoscopic image 40.

The first embodiment illustrates the lesion 42, which is merely an example. The region of interest (that is, the observation target region) to be gazed at by the doctor 16 may be an organ (for example, the duodenal papilla), a marked region, an artificial treatment tool (for example, an artificial clip), a treated region (for example, a region with a trace of removal of a polyp or the like), or the like.

The first display region 36 displays a moving image. The endoscopic image 40 displayed in the first display region 36 is one frame included in a moving image configured to include a plurality of frames along a time series. That is, the first display region 36 displays endoscopic images 40 of a plurality of frames at a specified frame rate (for example, 30 frames/second, 60 frames/second, or the like).

An example of the moving image to be displayed in the first display region 36 is a live-view moving image. The live-view moving image is merely an example, and the moving image may be a moving image that is temporarily stored in a memory or the like before being displayed, like a post-view moving image. Alternatively, each frame included in a recording moving image stored in the memory or the like may be reproduced and displayed in the first display region 36 as the endoscopic image 40.

On the screen 35, the second display region 38 is adjacent to the first display region 36 and is displayed in a lower right portion of the screen 35 when viewed from the front. The second display region 38 may be displayed at any position within the screen 35 of the display device 14, but is preferably displayed at a position that enables comparison with the endoscopic image 40.

The second display region 38 displays a probability map 45 including a segmentation image 44. The segmentation image 44 is an image region for identifying the position of the lesion 42, which is recognized by performing an object recognition process using an AI-based segmentation method on the endoscopic image 40, in the endoscopic image 40 (that is, an image displayed in a display format that can identify the position where the lesion 42 is most likely to be present in the endoscopic image 40).

The segmentation image 44 displayed in the second display region 38 is an image corresponding to the endoscopic image 40 and is referred to by the doctor 16 to identify the position of the lesion 42 in the endoscopic image 40.

While the segmentation image 44 is illustrated here, a bounding box is displayed instead of the segmentation image 44 in a case where the lesion 42 is recognized by performing an object recognition process using an AI-based bounding box method on the endoscopic image 40. Alternatively, the segmentation image 44 and the bounding box may be used in combination. The segmentation image 44 and the bounding box are merely examples, and any image may be used so long as the position of the lesion 42 appearing in the endoscopic image 40 can be identified.

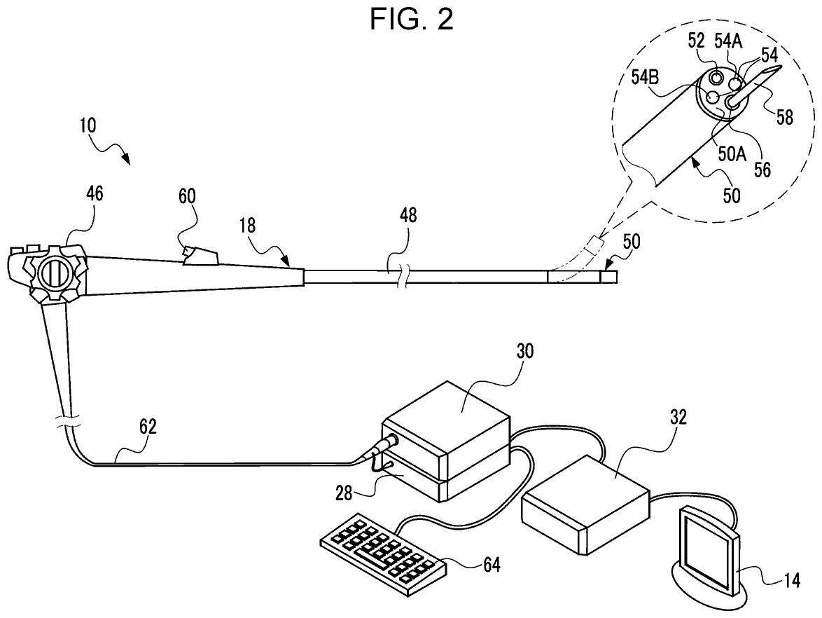

As an example, as illustrated in FIG. 2, the endoscope main body 18 includes an operation section 46 and an insertion section 48. The insertion section 48 partially bends in response to the operation section 46 being operated. When the doctor 16 (see FIG. 1) operates the operation section 46, the insertion section 48 is inserted into the large intestine 22 (see FIG. 1) while bending according to the shape of the large intestine 22.

The insertion section 48 has a tip portion 50 provided with a camera 52, an illumination device 54, and a treatment tool opening 56. The camera 52 and the illumination device 54 are provided on a tip surface 50A of the tip portion 50. While an example embodiment in which the camera 52 and the illumination device 54 are provided on the tip surface 50A of the tip portion 50 is given here, this is merely an example. The camera 52 and the illumination device 54 may be provided on a side surface of the tip portion 50 such that the endoscope 12 is configured as a side view endoscope.

The camera 52 is a device that performs imaging of the inside of the body (for example, the inside of the large intestine 22) of the subject 20 to acquire the endoscopic image 40 as a medical image. An example of the camera 52 is a CMOS camera. However, this is merely an example, and the camera 52 may be any other type of camera such as a CCD camera. The camera 52 is an example of a “module” according to the technology of the present disclosure.

The illumination device 54 has illumination windows 54A and 54B. The illumination device 54 emits the light 26 (see FIG. 1) through the illumination windows 54A and 54B. Types of the light 26 to be emitted from the illumination device 54 include, for example, visible light (for example, white light or the like) and invisible light (for example, near-infrared light or the like). The illumination device 54 further emits special light through the illumination windows 54A and 54B. Examples of the special light include light for BLI and/or light for LCI. The camera 52 performs imaging of the inside of the large intestine 22 using an optical method, with the inside of the large intestine 22 irradiated with the light 26 from the illumination device 54.

The treatment tool opening 56 is an opening for allowing a treatment tool 58 to protrude from the tip portion 50. The treatment tool opening 56 is also used as a suction port for sucking blood, bodily waste, and the like, and as a delivery port for delivering a fluid.

The operation section 46 has a treatment tool insertion port 60 formed therein, and the treatment tool 58 is inserted into the insertion section 48 through the treatment tool insertion port 60. The treatment tool 58 passes through the insertion section 48 and protrudes to the outside from the treatment tool opening 56. In the example illustrated in FIG. 2, a puncture needle is illustrated as the treatment tool 58 protruding from the treatment tool opening 56. While a puncture needle is illustrated as the treatment tool 58, this is merely an example, and the treatment tool 58 may be gripping forceps, a papillotomy knife, a snare, a catheter, a guide wire, a cannula, a puncture needle with a guide sheath, and/or the like.

The endoscope main body 18 is connected to the control device 28 and the light source device 30 through a universal cord 62. The control device 28 is connected to the medical support device 32 and a reception device 64. The medical support device 32 is further connected to the display device 14. That is, the control device 28 is connected to the display device 14 through the medical support device 32.

Since the medical support device 32 is illustrated here as an external device for extending the functions implemented by the control device 28, an example embodiment in which the control device 28 and the display device 14 are indirectly connected through the medical support device 32 is given here, although this is merely an example. For example, the display device 14 may be directly connected to the control device 28. In this case, for example, the control device 28 may be equipped with the functions of the medical support device 32, or the control device 28 may be equipped with a function of causing a server (not illustrated) to execute the same process as a process executed by the medical support device 32 (for example, a medical support process described below) and receiving and using a processing result obtained by the server.

The reception device 64 receives an instruction from the doctor 16 and outputs the received instruction to the control device 28 as an electrical signal. An example of the reception device 64 is a keyboard, a mouse, a touch panel, a foot switch, a microphone, and/or a remote operation device.

The control device 28 controls the light source device 30, transmits and receives various signals to and from the camera 52, and transmits and receives various signals to and from the medical support device 32.

The light source device 30 emits light under the control of the control device 28 and supplies the light to the illumination device 54. The illumination device 54 incorporates a light guide, and the light supplied from the light source device 30 is emitted from the illumination windows 54A and 54B through the light guide. The control device 28 causes the camera 52 to perform imaging, acquires the endoscopic image 40 (see FIG. 1) from the camera 52, and outputs the endoscopic image 40 to a specified output destination (for example, the medical support device 32).

The medical support device 32 performs various kinds of image processing on the endoscopic image 40 input from the control device 28. The medical support device 32 outputs the endoscopic image 40 on which the various kinds of image processing have been performed to a specified output destination (for example, the display device 14).

While an example embodiment has been described in which the endoscopic image 40 output from the control device 28 is output to the display device 14 through the medical support device 32, this is merely an example. For example, in another aspect, the control device 28 and the display device 14 may be connected to each other, and the endoscopic image 40 on which image processing has been performed by the medical support device 32 may be displayed on the display device 14 through the control device 28.

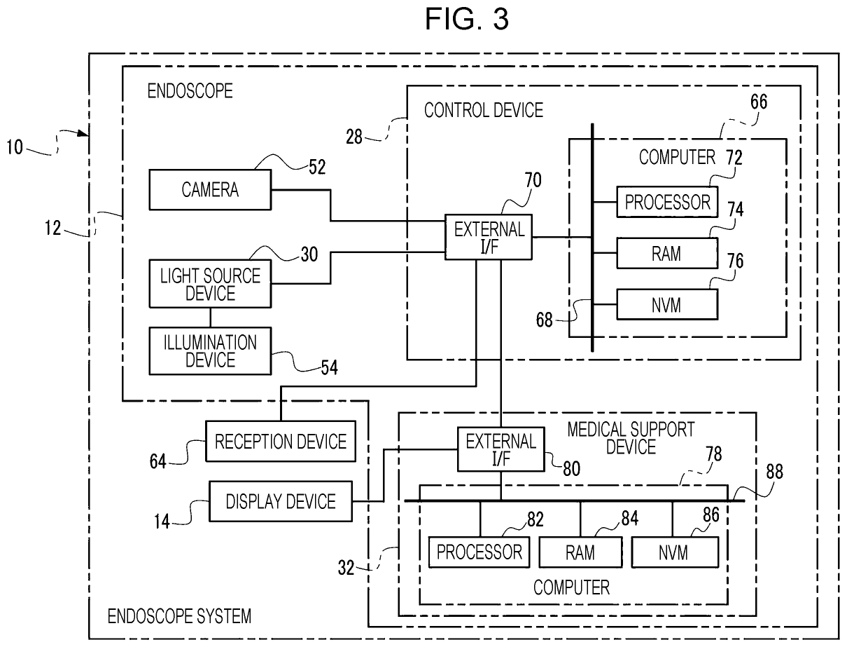

As an example, as illustrated in FIG. 3, the control device 28 includes a computer 66, a bus 68, and an external I/F 70. The computer 66 includes a processor 72, a RAM 74, and an NVM 76. The processor 72, the RAM 74, the NVM 76, and the external I/F 70 are connected to the bus 68.

For example, the processor 72 has at least one CPU and at least one GPU and controls the entire control device 28. The GPU operates under the control of the CPU and is responsible for performing various kinds of graphics-based processing, arithmetic operations using a neural network, and the like. The processor 72 may include one or more CPUs with integrated GPU functions, or may include one or more CPUs without integrated GPU functions. In the example illustrated in FIG. 3, the computer 66 is mounted with one processor 72. However, this is merely an example, and the computer 66 may be mounted with a plurality of processors 72.

The RAM 74 is a memory that temporarily stores information and is used as a work memory by the processor 72. The NVM 76 is a non-volatile storage device that stores various programs, various parameters, and the like. An example of the NVM 76 is a flash memory (for example, an EEPROM and/or an SSD). The flash memory is merely an example, and the NVM 76 may be any other non-volatile storage device such as an HDD, or a combination of two or more types of non-volatile storage devices.

The external I/F 70 handles transmission and reception of various kinds of information between the processor 72 and one or more devices (hereinafter also referred to as “first external devices”) external to the control device 28. An example of the external I/F 70 is a USB interface.

The camera 52 is connected to the external I/F 70 as one of the first external devices, and the external I/F 70 handles transmission and reception of various kinds of information between the camera 52 and the processor 72. The processor 72 controls the camera 52 through the external I/F 70. Further, the processor 72 acquires the endoscopic image 40 (see FIG. 1), which is obtained by imaging of the inside of the large intestine 22 (see FIG. 1) using the camera 52, through the external I/F 70.

The light source device 30 is connected to the external I/F 70 as one of the first external devices, and the external I/F 70 handles transmission and reception of various kinds of information between the light source device 30 and the processor 72. The light source device 30 supplies light to the illumination device 54 under the control of the processor 72. The illumination device 54 emits the light supplied from the light source device 30.

The reception device 64 is connected to the external I/F 70 as one of the first external devices, and the processor 72 acquires an instruction received by the reception device 64 through the external I/F 70 and executes a process corresponding to the acquired instruction.

The medical support device 32 includes a computer 78 and an external I/F 80. The computer 78 includes a processor 82, a RAM 84, and an NVM 86. The processor 82, the RAM 84, the NVM 86, and the external I/F 80 are connected to a bus 88. In the first embodiment, the medical support device 32 is an example of a “medical support device” according to the technology of the present disclosure, the computer 78 is an example of a “computer” according to the technology of the present disclosure, and the processor 82 is an example of a “processor” according to the technology of the present disclosure.

Since the hardware configuration (that is, the processor 82, the RAM 84, and the NVM 86) of the computer 78 is basically the same as the hardware configuration of the computer 66, the description of the hardware configuration of the computer 78 will be omitted here.

The external I/F 80 handles transmission and reception of various kinds of information between the processor 82 and one or more devices (hereinafter also referred to as “second external devices”) external to the medical support device 32. An example of the external I/F 80 is a USB interface.

The control device 28 is connected to the external I/F 80 as one of the second external devices. In the example illustrated in FIG. 3, the external I/F 70 of the control device 28 is connected to the external I/F 80. The external I/F 80 handles transmission and reception of various kinds of information between the processor 82 of the medical support device 32 and the processor 72 of the control device 28. For example, the processor 82 acquires the endoscopic image 40 (see FIG. 1) from the processor 72 of the control device 28 through the external I/Fs 70 and 80, and performs various kinds of image processing on the acquired endoscopic image 40.

The display device 14 is connected to the external I/F 80 as one of the second external devices. The processor 82 controls the display device 14 through the external I/F 80 to display various kinds of information (for example, the endoscopic image 40 and the like on which the various kinds of image processing have been performed) on the display device 14.

In an endoscopic examination, the doctor 16 determines whether the lesion 42 appearing in the endoscopic image 40 requires medical treatment, while checking the endoscopic image 40 through the display device 14, and performs medical treatment on the lesion 42, if necessary. The size of the lesion 42 is a determination factor important for determining whether medical treatment is necessary.

The recent development of machine learning has enabled the AI-based detection and classification of the lesion 42 based on the endoscopic image 40. Application of this technique makes it possible to measure the size of the lesion 42 from the endoscopic image 40. Accurately measuring the size of the lesion 42 and presenting the measurement result to the doctor 16 are very useful for the doctor 16 to perform medical treatment on the lesion 42.

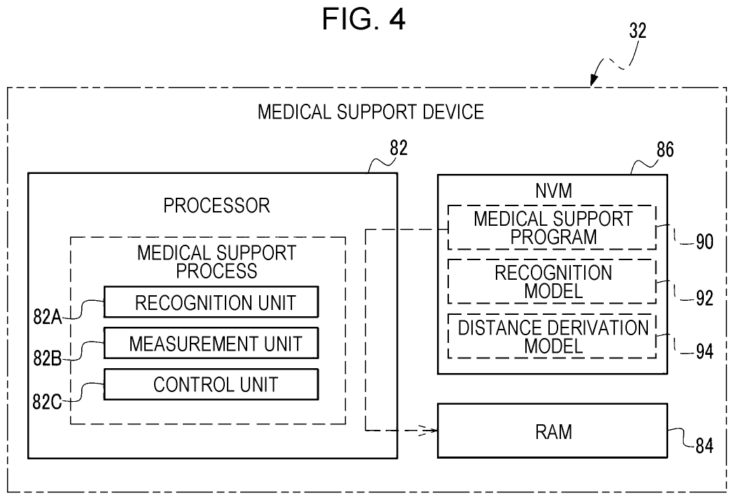

In view of such circumstances, in the first embodiment, as an example, as illustrated in FIG. 4, the processor 82 of the medical support device 32 performs a medical support process.

The NVM 86 stores a medical support program 90. The medical support program 90 is an example of a “program” according to the technology of the present disclosure. The processor 82 reads the medical support program 90 from the NVM 86 and executes the read medical support program 90 on the RAM 84 to perform the medical support process. The medical support process is implemented by the processor 82 operating as a recognition unit 82A, a measurement unit 82B, and a control unit 82C in accordance with the medical support program 90 executed on the RAM 84.

The NVM 86 stores a recognition model 92 and a distance derivation model 94. As described in detail below, the recognition model 92 is used by the recognition unit 82A, and the distance derivation model 94 is used by the measurement unit 82B. In the first embodiment, the recognition model 92 is an example of an “AI” according to the technology of the present disclosure.

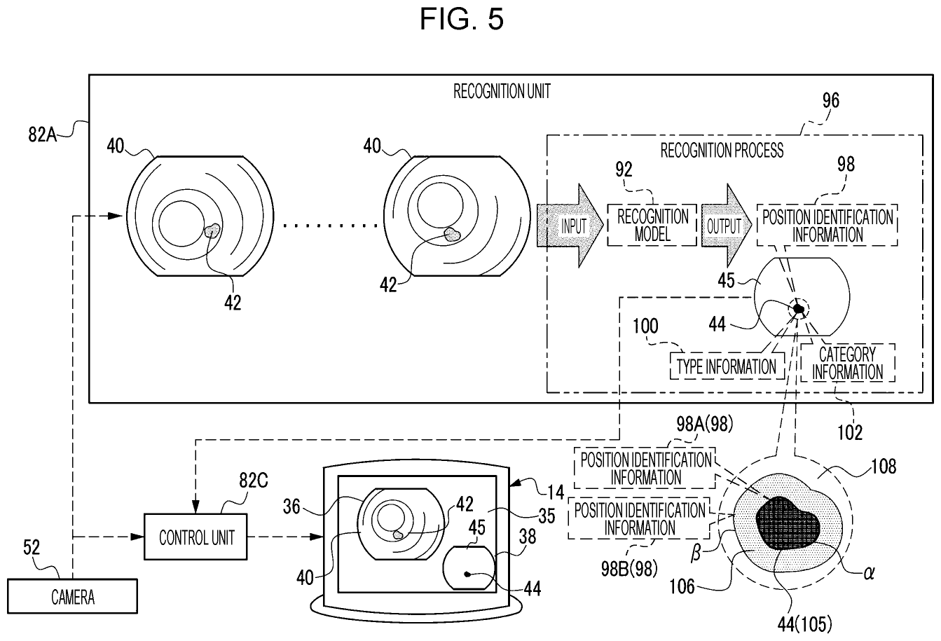

As an example, as illustrated in FIG. 5, the recognition unit 82A and the control unit 82C acquire an endoscopic image 40, which is generated through imaging performed by the camera 52 in accordance with an imaging frame rate (for example, several tens of frames/second), from the camera 52 on a frame-by-frame basis.

The control unit 82C displays the endoscopic image 40 in the first display region 36 as a live view image. That is, each time the control unit 82C acquires an endoscopic image 40 from the camera 52 on a frame-by-frame basis, the control unit 82C sequentially displays the acquired endoscopic image 40 in the first display region 36 in accordance with a display frame rate (for example, several tens of frames/second).

The recognition unit 82A uses the endoscopic image 40 acquired from the camera 52 to recognize the lesion 42 in the endoscopic image 40. That is, the recognition unit 82A performs a recognition process 96 on the endoscopic image 40 acquired from the camera 52 to recognize the characteristics of the lesion 42 appearing in the endoscopic image 40. The recognition unit 82A recognizes, as the characteristics of the lesion 42, the shape of the lesion 42, the type of the lesion 42, the category of the lesion 42 (for example, pedunculated, sub-pedunculated, sessile, superficial elevated, superficial flat, superficial depressed, and the like), and the clarity of the contour of the lesion 42. The recognition of the shape of the lesion 42 and the clarity of the contour of the lesion 42 is implemented by recognizing the position of the lesion 42 in the endoscopic image 40 (that is, the position of the lesion 42 appearing in the endoscopic image 40).

Each time the recognition unit 82A acquires the endoscopic image 40, the recognition process 96 is performed on the acquired endoscopic image 40. The recognition process 96 is a process for recognizing the lesion 42 by a method using AI. In the first embodiment, for example, an object recognition process using an AI-based segmentation method (for example, semantic segmentation, instance segmentation, and/or panoptic segmentation) is used as the recognition process 96.

A process using the recognition model 92 is performed as the recognition process 96. The recognition model 92 is a trained model for object recognition using an AI-based segmentation method. An example of the trained model for object recognition using an AI-based segmentation method is a model for semantic segmentation. An example of the model for semantic segmentation is an encoder-decoder structure model. An example of the encoder-decoder structure model is a U-Net model or an HRNet model.

The recognition model 92 is optimized by training a neural network through machine learning using first training data. The first training data is a dataset including a plurality of pieces of data (that is, data for a plurality of frames) in which first example data and first ground-truth data are associated with each other.

The first example data is an image corresponding to the endoscopic image 40. The first ground-truth data is ground-truth data (that is, an annotation) for the first example data. An example of the first ground-truth data is an annotation for identifying the position, type, and category of a lesion appearing in an image used as the first example data.

The recognition unit 82A acquires the endoscopic image 40 from the camera 52 and inputs the acquired endoscopic image 40 to the recognition model 92. Accordingly, each time the endoscopic image 40 is input, the recognition model 92 identifies, as the position of the lesion 42 appearing in the input endoscopic image 40, the position of the segmentation image 44 identified by the segmentation method and outputs position identification information 98 that can identify the position of the segmentation image 44. Examples of the position identification information 98 include coordinates for identifying the segmentation image 44 in the endoscopic image 40. Each time the endoscopic image 40 is input, the recognition model 92 further recognizes the type of the lesion 42 appearing in the input endoscopic image 40 and outputs type information 100 indicating the recognized type. Each time the endoscopic image 40 is input, the recognition model 92 further recognizes the category of the lesion 42 appearing in the input endoscopic image 40 and outputs category information 102 indicating the recognized category. The segmentation image 44 is associated with the position identification information 98, the type information 100, and the category information 102.

The control unit 82C displays, for each endoscopic image 40, the probability map 45 indicating the distribution of the position of the lesion 42 in the second display region 38 in accordance with the segmentation image 44 and the position identification information 98. The probability map 45 is a map in which the distribution of the position of the lesion 42 in the endoscopic image 40 is expressed in terms of a probability, which is an example of a measure of the likelihood. The probability map 45 is obtained by the recognition unit 82A for each endoscopic image 40 from the recognition model 92. The probability map 45 is typically referred to also as a reliability map, a certainty map, or the like.

The probability map 45 displayed in the second display region 38 is updated in accordance with the display frame rate applied to the first display region 36. That is, the display of the probability map 45 in the second display region 38 (that is, the display of the segmentation image 44) is updated in synchronization with the display timing of the endoscopic image 40 displayed in the first display region 36. This allows the doctor 16 to grasp the schematic position of the lesion 42 in the endoscopic image 40 displayed in the first display region 36 by referring to the probability map 45 displayed in the second display region 38 while observing the endoscopic image 40 displayed in the first display region 36. In the first embodiment, the probability map 45 is an example of a “probability map” according to the technology of the present disclosure.

In the probability map 45, the position where the lesion 42 is present is segmented according to probabilities. In the example illustrated in FIG. 5, the probability map 45 is divided into three closed regions, namely, a first segment region 105, a second segment region 106, and a third segment region 108, in accordance with threshold values α and β. The threshold values α and β have a relationship of “α>β”. In the probability map 45, a closed region having a probability greater than or equal to the threshold value a is the first segment region 105, and the first segment region 105 corresponds to the segmentation image 44. In the probability map 45, a closed region having a probability greater than or equal to the threshold value β and less than the threshold value α is the second segment region 106. In the probability map 45, a closed region having a probability less than the threshold value β is the third segment region 108. An example of the threshold values α and β is values determined in accordance with an instruction received by the reception device 64 or values determined in accordance with various conditions. The threshold value α and/or β may be a fixed value or a variable value that is changed in accordance with a given instruction and various conditions.

In the first embodiment, the threshold values α and β are an example of a “plurality of threshold values” according to the technology of the present disclosure. In the first embodiment, the first segment region 105, the second segment region 106, and the third segment region 108 are an example of a “plurality of segment regions” according to the technology of the present disclosure. In the first embodiment, the first segment region 105 is a “closed region” and a “first segment region” according to the technology of the present disclosure, and the second segment region 106 is an example of a “closed region” and a “second segment region” according to the technology of the present disclosure.

The position identification information 98 is roughly divided into position identification information 98A and position identification information 98B. The position identification information 98A is associated with the segmentation image 44 (that is, the first segment region 105), and the position identification information 98B is associated with the second segment region 106. The position identification information 98A is a plurality of coordinates for identifying the position of the contour of the segmentation image 44 in the endoscopic image 40. The position identification information 98B is a plurality of coordinates for identifying the position of the contour (for example, the outer contour and the inner contour) of the second segment region 106 in the endoscopic image 40.

While the threshold values α and β are illustrated here, the technology of the present disclosure is not limited to these, and three or more threshold values may be used. While the first segment region 105, the second segment region 106, and the third segment region 108 are illustrated here, the technology of the present disclosure is not limited to these, and one segment region or three or more segment regions may be used. The number of segment regions can be changed according to the number of threshold values.

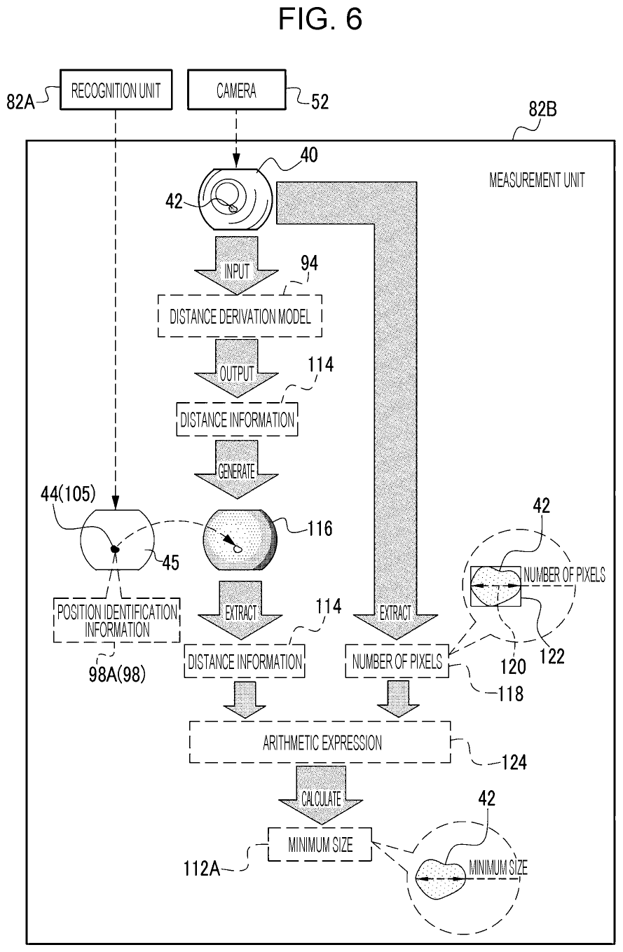

As an example, as illustrated in FIG. 6, the measurement unit 82B measures a minimum size 112A of the lesion 42, based on the endoscopic image 40 acquired from the camera 52. To implement the measurement of the minimum size 112A of the lesion 42, the measurement unit 82B acquires distance information 114 of the lesion 42, based on the endoscopic image 40 acquired from the camera 52. The distance information 114 is information indicating the distance from the camera 52 (that is, the observation position) to the intestinal wall 24 (see FIG. 1) including the lesion 42. While the distance from the camera 52 to the intestinal wall 24 including the lesion 42 is illustrated here, this is merely an example. Instead of the distance, a numerical value indicating the depth from the camera 52 to the intestinal wall 24 including the lesion 42 (for example, a plurality of numerical values defining depths in a stepwise manner (for example, numerical values in several steps to several tens of steps)) may be used.

The distance information 114 is acquired for each of all the pixels constituting the endoscopic image 40. The distance information 114 may be acquired for each block (for example, a pixel group constituted by several to several hundreds of pixels), which is larger than a pixel in the endoscopic image 40.

The measurement unit 82B acquires the distance information 114 by, for example, deriving the distance information 114 by using an AI-based method. In the first embodiment, the distance derivation model 94 is used to derive the distance information 114.

The distance derivation model 94 is optimized by training the neural network through machine learning using second training data. The second training data is a dataset including a plurality of pieces of data (that is, data for a plurality of frames) in which second example data and second ground-truth data are associated with each other.

The second example data is an image corresponding to the endoscopic image 40. The second ground-truth data is ground-truth data (that is, an annotation) for the second example data. An example of the second ground-truth data is an annotation for identifying a distance corresponding to each pixel appearing in an image used as the second example data.

The measurement unit 82B acquires the endoscopic image 40 from the camera 52 and inputs the acquired endoscopic image 40 to the distance derivation model 94. As a result, the distance derivation model 94 outputs the distance information 114 on a pixel-by-pixel basis in the input endoscopic image 40. That is, in the measurement unit 82B, information indicating the distance from the position of the camera 52 (for example, the position of the image sensor, the objective lens, or the like mounted in the camera 52) to the intestinal wall 24 appearing in the endoscopic image 40 is output from the distance derivation model 94 as the distance information 114 on a pixel-by-pixel basis in the endoscopic image 40.

The measurement unit 82B generates a distance image 116, based on the distance information 114 output from the distance derivation model 94. The distance image 116 is an image in which the distance information 114 is distributed in units of pixels included in the endoscopic image 40.

The measurement unit 82B acquires the position identification information 98A assigned to the segmentation image 44 on the probability map 45 obtained by the recognition unit 82A. The measurement unit 82B refers to the position identification information 98A and extracts, from the distance image 116, the distance information 114 corresponding to the position identified from the position identification information 98A. Examples of the distance information 114 extracted from the distance image 116 include the distance information 114 corresponding to the position (for example, centroid) of the lesion 42, and the statistical value (for example, median value, mean value, or mode value) of the distance information 114 for a plurality of pixels (for example, all the pixels) included in the lesion 42.

The measurement unit 82B extracts the number of pixels 118 from the endoscopic image 40. The number of pixels 118 is the number of pixels on a line segment 120 crossing an image region at a position identified from the position identification information 98A (that is, an image region indicating the lesion 42) within an entire image region of the endoscopic image 40 input to the distance derivation model 94. An example of the line segment 120 is the longest line segment parallel to the long sides of a circumscribed rectangular frame 122 of the image region indicating the lesion 42. The line segment 120 is merely an example. Instead of the line segment 120, the longest line segment parallel to the short sides of the circumscribed rectangular frame 122 of the image region indicating the lesion 42 may be used.

In the first embodiment, the line segment 120 is an example of a “long side of the observation target region” according to the technology of the present disclosure, and the longest line segment parallel to the short sides of the circumscribed rectangular frame 122 of the image region indicating the lesion 42 is an example of a “short side of the observation target region” according to the technology of the present disclosure.

The measurement unit 82B calculates the minimum size 112A of the lesion 42 in real space, based on the distance information 114 extracted from the distance image 116 and the number of pixels 118 extracted from the endoscopic image 40. The minimum size 112A refers to, for example, the minimum size expected to be the size of the lesion 42 in real space. In the example illustrated in FIG. 6, the size of the first segment region 105, which is the smallest segment region among the plurality of segment regions, in real space, that is, the size of the segmentation image 44 in real space (that is, the actual size in the body), is illustrated as an example of the minimum size 112A.

The minimum size 112A is calculated using an arithmetic expression 124. The measurement unit 82B inputs the distance information 114 extracted from the distance image 116 and the number of pixels 118 extracted from the endoscopic image 40 to the arithmetic expression 124. The arithmetic expression 124 is an arithmetic expression in which the distance information 114 and the number of pixels 118 are independent variables and the minimum size 112A is a dependent variable. The arithmetic expression 124 outputs the minimum size 112A corresponding to the input distance information 114 and the input number of pixels 118.

While the length of the lesion 42 in real space is illustrated as the minimum size 112A, the technology of the present disclosure is not limited to this. The minimum size 112A may be the surface area or volume of the lesion 42 in real space. In this case, for example, as the arithmetic expression 124, an arithmetic expression is used in which the number of pixels in an entire image region indicating the lesion 42 and the distance information 114 are independent variables and the surface area or volume of the lesion 42 in real space is a dependent variable.

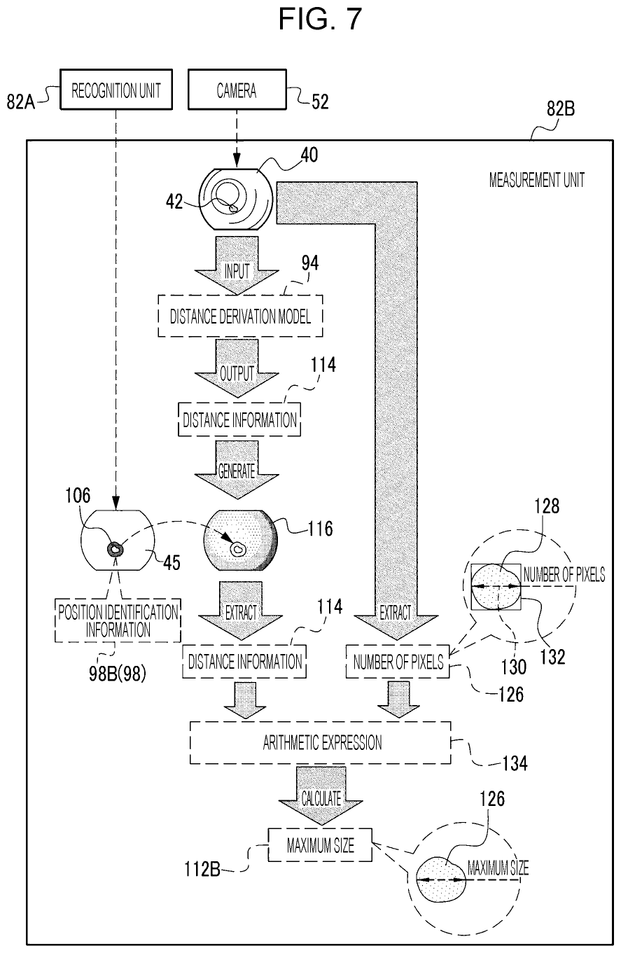

As an example, as illustrated in FIG. 7, the measurement unit 82B measures a maximum size 112B of the lesion 42, based on the endoscopic image 40 acquired from the camera 52. The maximum size 112B is measured in a manner similar to that for the minimum size 112A. The minimum size 112A is measured using the segmentation image 44 and the position identification information 98A, whereas the maximum size 112B is measured using the second segment region 106 and the position identification information 98B. A more detailed description is given below.

The measurement unit 82B acquires the position identification information 98B assigned to the second segment region 106 on the probability map 45 obtained by the recognition unit 82A. The measurement unit 82B refers to the position identification information 98B and extracts, from the distance image 116, the distance information 114 corresponding to the position identified from the position identification information 98B. Examples of the distance information 114 extracted from the distance image 116 include the distance information 114 corresponding to an annular closed region (for example, inner contour and/or outer contour) identified from the position identification information 98B in the endoscopic image 40, and the statistical value (for example, median value, mean value, or mode value) of the distance information 114 for a plurality of pixels included in an annular closed region identified from the position identification information 98B in the endoscopic image 40 (for example, all the pixels constituting the annular closed region, all the pixels constituting the inner contour of the annular closed region, or all the pixels constituting the outer contour of the annular closed region).

The measurement unit 82B extracts an image region 128 from the endoscopic image 40. The image region 128 is a region enclosed by the outer contour of the annular closed region identified from the position identification information 98B within the entire image region of the endoscopic image 40 input to the distance derivation model 94. Then, the measurement unit 82B extracts the number of pixels 126 from the image region 128. The number of pixels 126 is the number of pixels on a line segment 130 crossing the image region 128. An example of the line segment 130 is the longest line segment parallel to the long sides of a circumscribed rectangular frame 132 of the image region 128. The line segment 130 is merely an example. Instead of the line segment 130, the longest line segment parallel to the short sides of the circumscribed rectangular frame 132 of the image region 128 may be used.

The measurement unit 82B calculates the maximum size 112B of the lesion 42 in real space, based on the distance information 114 extracted from the distance image 116 and the number of pixels 126 extracted from the endoscopic image 40. The maximum size 112B refers to, for example, the maximum size expected to be the size of the lesion 42 in real space. In the example illustrated in FIG. 7, the size of the second segment region 106, which is an outer region with respect to the first segment region 105 among the plurality of segment regions, in real space (that is, the actual size in the body) is illustrated as an example of the maximum size 112B.

The maximum size 112B is calculated using an arithmetic expression 134. The measurement unit 82B inputs the distance information 114 extracted from the distance image 116 and the number of pixels 126 extracted from the endoscopic image 40 to the arithmetic expression 134. The arithmetic expression 134 is an arithmetic expression in which the distance information 114 and the number of pixels 126 are independent variables and the maximum size 112B is a dependent variable. The arithmetic expression 134 outputs the maximum size 112B corresponding to the input distance information 114 and the input number of pixels 126.

While the length of the second segment region 106 in real space is illustrated as the maximum size 112B, the technology of the present disclosure is not limited to this. The maximum size 112B may be the surface area or volume of the second segment region 106 in real space. In this case, for example, as the arithmetic expression 134, an arithmetic expression is used in which the number of pixels in an entire image region indicating the second segment region 106 and the distance information 114 are independent variables and the surface area or volume of the second segment region 106 in real space is a dependent variable.

In the example illustrated in FIGS. 6 and 7, two sizes, namely, the minimum size 112A and the maximum size 112B, are measured by the measurement unit 82B. However, this is merely an example, and three or more sizes may be measured by the measurement unit 82B. In this case, it is sufficient that three or more segment regions be obtained using three or more threshold values and the size of each segment region be measured in the manner described above.

In the first embodiment, the minimum size 112A and the maximum size 112B are an example of “a plurality of first sizes of the observation target region”. In the first embodiment, the minimum size 112A is an example of a “lower limit value of the range” according to the technology of the present disclosure. In the first embodiment, the maximum size 112B is an example of an “upper limit value of the range” according to the technology of the present disclosure.

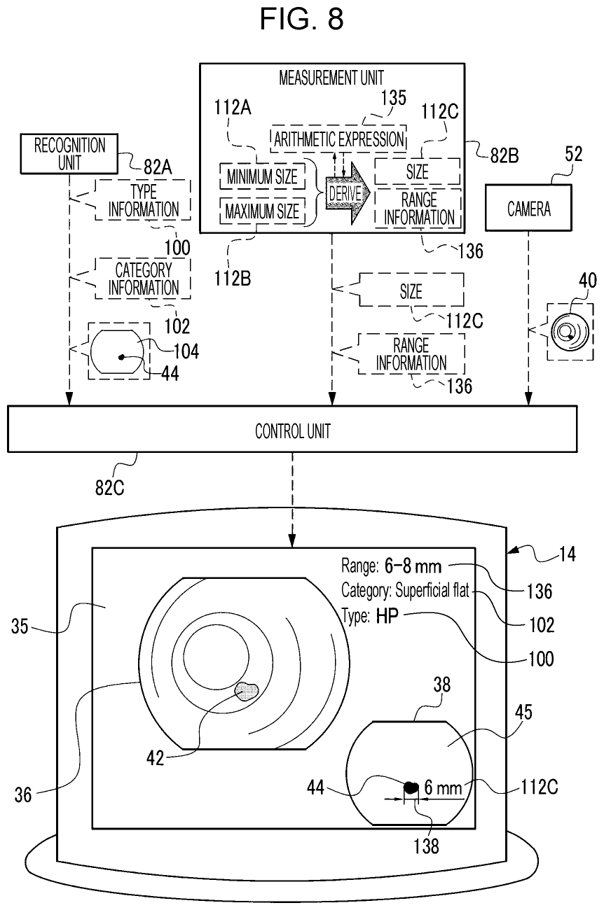

As an example, as illustrated in FIG. 8, the measurement unit 82B measures the size 112C of the lesion 42 by using the minimum size 112A and the maximum size 112B measured based on the endoscopic image 40. In the first embodiment, the “size 112C” is an example of a “size of the observation target region” according to the technology of the present disclosure.

The size 112C is a size corresponding to the characteristics of the lesion 42. The characteristics of the lesion 42 refer to, for example, the shape of the lesion 42, the type of the lesion 42, the category of the lesion 42, and the clarity of the contour of the lesion 42. The shape of the lesion 42 and the clarity of the contour of the lesion 42 (for example, the range of variation in the contour of the lesion 42 due to, for example, how the lesion 42 appears in the endoscopic image 40) are identified from the minimum size 112A and the maximum size 112B, the type of the lesion 42 is identified from the type information 100, and the category of the lesion 42 is identified from the category information 102.

In the first embodiment, the minimum size 112A and the maximum size 112B are sizes corresponding to the characteristics of the lesion 42, and the measurement of the size 112C is implemented by deriving the size 112C from the minimum size 112A and the maximum size 112B.

The size 112C is calculated from an arithmetic expression 135 in which the minimum size 112A and the maximum size 112B are independent variables and the size 112C is a dependent variable. Alternatively, the size 112C may be derived from a table having the minimum size 112A and the maximum size 112B as inputs and the size 112C as an output.

The size 112C is, for example, the mean value of the minimum size 112A and the maximum size 112B. The mean value is merely an example, and the size 112C is any representative value of the minimum size 112A and the maximum size 112B. An example of the representative value is a statistical value. The statistical value refers to the maximum value, the minimum value, the median value, the mean value, the variance value, and/or the like. In the first embodiment, a size 112C is an example of a “representative value of the plurality of first sizes” according to the technology of the present disclosure.

In the known technique of the related art, in some cases, the size of the lesion 42 in one frame is not uniquely defined due to how the lesion 42 appears in the endoscopic image 40, the shape of the lesion 42 appearing in the endoscopic image 40, the structure of the AI, an insufficient amount of learning in relation to the AI, and/or the like. For example, the measured size may have a range (that is, a range of variation), or a plurality of sizes may be measured.

Accordingly, the measurement unit 82B derives range information 136, based on the minimum size 112A and the maximum size 112B. The range information 136 is information indicating the range of the size of the lesion 42 in real space (hereinafter also referred to as the “actual size of the lesion 42”). The range of the actual size of the lesion 42 is identified based on the first segment region 105 (that is, the segmentation image 44) and the second segment region 106. For example, the range of the actual size of the lesion 42 is identified by using the minimum size 112A and the maximum size 112B.

The measurement unit 82B derives, as the range information 136, information (for example, text information or an image) that can identify both the maximum size 112B and the minimum size 112A. This is merely an example, and the measurement unit 82B may derive, as the range information 136, information using the absolute value of the difference between the maximum size 112B, which is the upper limit value of the range of the actual size of the lesion 42, and the minimum size 112A, which is the lower limit value of the range of the actual size of the lesion 42, together with or instead of information that can identify both the maximum size 112B and the minimum size 112A.

The control unit 82C acquires the size 112C from the measurement unit 82B. Further, the control unit 82C acquires the probability map 45 from the recognition unit 82A. The control unit 82C displays the probability map 45 acquired from the recognition unit 82A in the second display region 38. Then, the control unit 82C displays the size 112C acquired from the measurement unit 82B on the probability map 45. For example, the size 112C is superimposed and displayed on the probability map 45. The superimposed display is merely an example, and embedded display may be used.

The control unit 82C displays a dimension line 138 on the probability map 45 as a mark that can identify which portion of the lesion 42 has a size (that is, a length) corresponding to the size 112C to be displayed on the probability map 45. For example, the control unit 82C acquires the position identification information 98A from the recognition unit 82A, and the dimension line 138 is generated based on the position identification information 98A and displayed. It is sufficient that, for example, the dimension line 138 be generated in a manner similar to that for the generation of the line segment 120 (that is, in a manner similar to that using the circumscribed rectangular frame 122).

The control unit 82C acquires the type information 100 and the category information 102 from the recognition unit 82A and displays, on the screen 35, the type of the lesion 42 indicated by the type information 100 and the category of the lesion 42 indicated by the category information 102. In the example illustrated in FIG. 8, the type information 100 and category information 102 are displayed in text format on the screen 35. The type information 100 and the category information 102 may be displayed on the screen 35 in a format (for example, an image or the like) other than a text format.

The control unit 82C acquires the range information 136 from the measurement unit 82B and displays, on the screen 35, the range of the actual size of the lesion 42 indicated by the range information 136. In the example illustrated in FIG. 8, the range information 136 is displayed in text format on the screen 35. The range information 136 may be displayed on the screen 35 in a format (for example, an image or the like) other than a text format. For example, a curve obtained by offsetting the outer contour of the segmentation image 44 outward by the range indicated by the range information 136 may be displayed on the outer periphery of the segmentation image 44, or the range information 136 may be displayed as an image along the outer periphery of the segmentation image 44 in a specific display format (for example, a display format distinguishable from the other regions on the probability map 45).

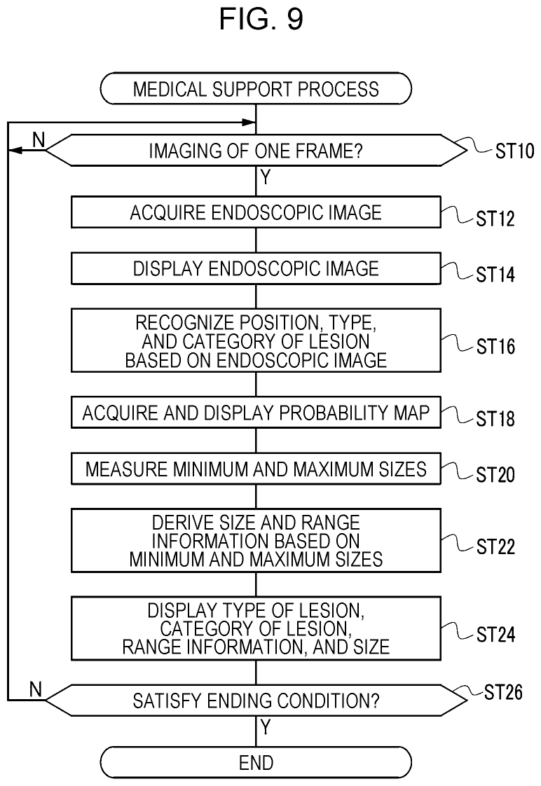

Next, the operation of a portion, according to the technology of the present disclosure, of the endoscope system 10 will be described with reference to FIG. 9. The flow of the medical support process illustrated in FIG. 9 is an example of a “medical support method” according to the technology of the present disclosure.

In the medical support process illustrated in FIG. 9, first, in step ST10, the recognition unit 82A determines whether imaging of one frame has been performed in the large intestine 22 by the camera 52. If imaging of one frame has not been performed in the large intestine 22 by the camera 52 in step ST10, the determination is negative, and the determination of step ST10 is performed again. If imaging of one frame has been performed in the large intestine 22 by the camera 52 in step ST10, the determination is affirmative, and the medical support process proceeds to step ST12.

In step ST12, the recognition unit 82A and the control unit 82C acquire an endoscopic image 40 corresponding to one frame, which is obtained by imaging of the large intestine 22 using the camera 52 (see FIG. 5). For convenience of description, it is assumed here that the lesion 42 appears in the endoscopic image 40. After the processing of step ST12 is performed, the medical support process proceeds to step ST14.

In step ST14, the control unit 82C displays the endoscopic image 40 acquired in step ST12 in the first display region 36 (see FIGS. 1, 5, and 8). After the processing of step ST14 is performed, the medical support process proceeds to step ST16.

In step ST16, the recognition unit 82A performs the recognition process 96 using the endoscopic image 40 acquired in step ST12 to recognize the position, type, and category of the lesion 42 in the endoscopic image 40, and acquires the position identification information 98, the type information 100, and the category information 102 (see FIG. 5). After the processing of step ST16 is performed, the medical support process proceeds to step ST18.

In step ST18, the recognition unit 82A acquires the probability map 45 from the recognition model 92 used to recognize the position, type, and category of the lesion 42 in step ST16. Then, the control unit 82C displays the probability map 45, which is acquired from the recognition model 92 by the recognition unit 82A, in the second display region 38 (see FIGS. 1, 5, and 8). After the processing of step ST18 is performed, the medical support process proceeds to step ST20.

In step ST20, the measurement unit 82B measures the minimum size 112A of the lesion 42, based on the endoscopic image 40 used in step ST16 and the position identification information 98A obtained through the recognition process 96 performed in step ST16 (see FIG. 6). Further, the measurement unit 82B measures the maximum size 112B of the lesion 42, based on the endoscopic image 40 used in step ST16 and the position identification information 98B obtained through the recognition process 96 performed in step ST16 (see FIG. 7). After the processing of step ST20 is performed, the medical support process proceeds to step ST22.

In step ST22, the measurement unit 82B derives the size 112C and the range information 136, based on the minimum size 112A and the maximum size 112B measured in step ST20. After the processing of step ST22 is performed, the medical support process proceeds to step ST24.

In step ST24, the control unit 82C displays, on the screen 35, the type of the lesion 42 indicated by the type information 100 acquired in step ST16 and the category of the lesion 42 indicated by the category information 102 acquired in step ST16 (see FIG. 8). Further, the control unit 82C displays the range indicated by the range information 136 derived in step ST22 on the screen 35 (see FIG. 8). Further, the control unit 82C displays the size 112C derived in step ST22 on the probability map 45. After the processing of step ST24 is performed, the medical support process proceeds to step ST26.

In step ST26, the control unit 82C determines whether a condition for ending the medical support process is satisfied. An example of the condition for ending the medical support process is a condition in which an instruction to end the medical support process is given to the endoscope system 10 (for example, a condition in which the instruction to end the medical support process is received by the reception device 64).

If the condition for ending the medical support process is not satisfied in step ST26, the determination is negative, and the medical support process returns to step ST10. If the condition for ending the medical support process is satisfied in step ST26, the determination is affirmative, and the medical support process ends.

As described above, in the endoscope system 10 according to the first embodiment, the recognition unit 82A uses the endoscopic image 40 to recognize the lesion 42 appearing in the endoscopic image 40. Further, the measurement unit 82B measures the size 112C of the lesion 42, based on the endoscopic image 40. Then, the control unit 82C displays the size 112C on the screen 35.

The size 112C is a size corresponding to the characteristics of the lesion 42. The characteristics of the lesion 42 refer to, for example, the shape of the lesion 42, the type of the lesion 42, the category of the lesion 42, and the clarity of the contour of the lesion 42. This allows the doctor 16 to more accurately grasp the size 112C of the lesion 42 appearing in the endoscopic image 40 than in a case where the size of the lesion 42 appearing in the endoscopic image 40 is measured without any consideration of the characteristics of the lesion 42. This also allows the doctor 16 to more accurately grasp the size 112C of the lesion 42 appearing in the endoscopic image 40 than in a case where the size of the lesion 42 appearing in the endoscopic image 40 is measured without any consideration of the shape of the lesion 42, the type of the lesion 42, the category of the lesion 42, and the clarity of the contour of the lesion 42.

In the endoscope system 10 according to the first embodiment, the recognition unit 82A recognizes the characteristics of the lesion 42, based on the endoscopic image 40. Accordingly, the characteristics of the lesion 42 appearing in the endoscopic image 40 can be accurately identified.

In the endoscope system 10 according to the first embodiment, furthermore, the size 112C of a region corresponding to the line segment 120 is measured and displayed on the screen 35. The line segment 120 is the longest line segment parallel to the long sides of the circumscribed rectangular frame 122 of the image region indicating the lesion 42. This enables the doctor 16 to grasp the length, in real space, of the longest region crossing the lesion 42 along the longest line segment parallel to the long sides of the circumscribed rectangular frame 122 of the image region indicating the lesion 42.

In the endoscope system 10 according to the first embodiment, furthermore, the lesion 42 is recognized by a method using the recognition model 92, and the size 112C is measured based on the probability map 45 obtained from the recognition model 92. Accordingly, the size 112C of the lesion 42 appearing in the endoscopic image 40 can be accurately measured.

In the endoscope system 10 according to the first embodiment, furthermore, measurement is performed based on closed regions obtained by dividing the probability map 45 in accordance with the threshold values a and B. Accordingly, the position of the lesion 42 in the endoscopic image 40 can be accurately identified. Thus, the size 112C (that is, the size in real space) of the lesion 42 appearing in the endoscopic image 40 can be accurately measured.

In the endoscope system 10 according to the first embodiment, furthermore, the size 112C of the lesion 42 is measured based on the first segment region 105 and the second segment region 106 obtained by dividing the probability map 45 in accordance with the threshold values a and B. Accordingly, even when the contour of the lesion 42 appearing in the endoscopic image 40 is unclear due to body motion, the movement of the camera 52, and/or the like, the size 112C of the lesion 42 can be accurately measured.

In the endoscope system 10 according to the first embodiment, furthermore, the range information 136 is measured based on the minimum size 112A and the maximum size 112B and is displayed on the screen 35. This allows the doctor 16 to accurately grasp the range of variation in the actual size of the lesion 42 appearing in the endoscopic image 40. That is, the doctor 16 can accurately grasp the lower limit value and the upper limit value of the actual size of the lesion 42 appearing in the endoscopic image 40. As a result, the doctor 16 can predict that the actual size of the lesion 42 is likely to fall within the range indicated by the range information 136.

In the endoscope system 10 according to the first embodiment, furthermore, a representative value (for example, maximum value, minimum value, mean value, median value, variance value, and/or the like) of the minimum size 112A and the maximum size 112B is used as the size 112C to be displayed on the screen 35. This allows the doctor 16 to grasp the actual size of the lesion 42 without ambiguity, as compared to a case where a plurality of sizes are displayed on the screen 35.

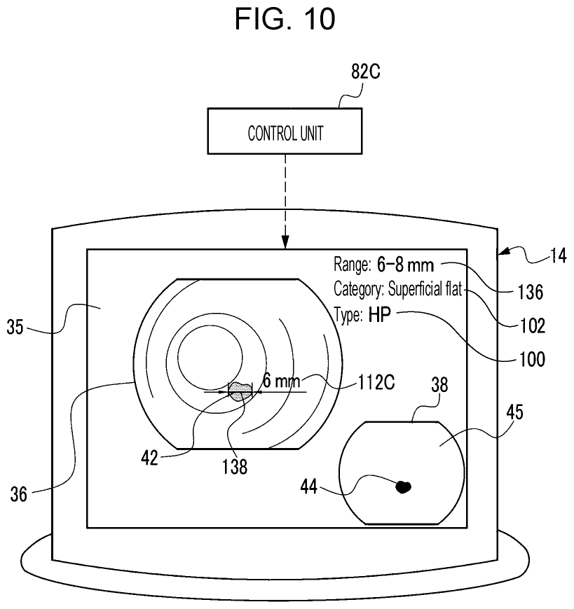

While the first embodiment described above provides an example embodiment in which the size 112C and the dimension line 138 are displayed on the probability map 45, the technology of the present disclosure is not limited to this. For example, as illustrated in FIG. 10, the size 112C and the dimension line 138 may be displayed on the endoscopic image 40. The size 112C and/or the dimension line 138 may be superimposed and displayed on the endoscopic image 40 by using alpha blending, or may be displayed on the endoscopic image 40 such that the display format such as the display position, the display size, and/or the display color of the size 112C and/or the dimension line 138 can be changed in accordance with an instruction received by the reception device 64.

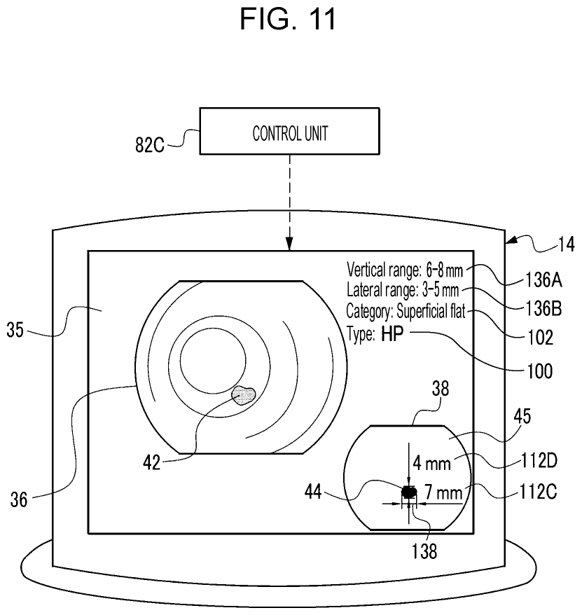

While the first embodiment described above provides an example embodiment in which the size 112C is measured as the length, in real space, of the longest region crossing the lesion 42 along the line segment 120, the technology of the present disclosure is not limited to this. For example, as illustrated in FIG. 11, a size 112D of a region corresponding to the longest line segment parallel to the short sides of the circumscribed rectangular frame 122 of the image region indicating the lesion 42 may be measured and displayed on the screen 35. In this case, the doctor 16 can grasp the length, in real space, of the longest region crossing the lesion 42 along the longest line segment parallel to the short sides of the circumscribed rectangular frame 122 of the image region indicating the lesion 42.

The actual size of the lesion 42, which is measured in relation to the radius and/or diameter of a circumcircle of the image region indicating the lesion 42, may be displayed on the screen 35. In this case, the doctor 16 can grasp the actual size of the lesion 42 in relation to the radius and/or diameter of the circumcircle of the image region indicating the lesion 42. Second Embodiment

While the first embodiment described above provides an example embodiment in which an image of the lesion 42 is captured by the camera 52 when no obstacle is present between the camera 52 and the lesion 42, the second embodiment describes a case in which when an obstacle is present between the camera 52 and the lesion 42, an image of a region (that is, the inside of the large intestine 22) including the obstacle and a portion of the lesion 42 that is not blocked by the obstacle is captured by the camera 52. In the following, for convenience of description, the same components as those in the first embodiment described above are denoted by the same reference numerals as in the first embodiment, and descriptions thereof will be omitted. The differences from the first embodiment described above will be mainly described.

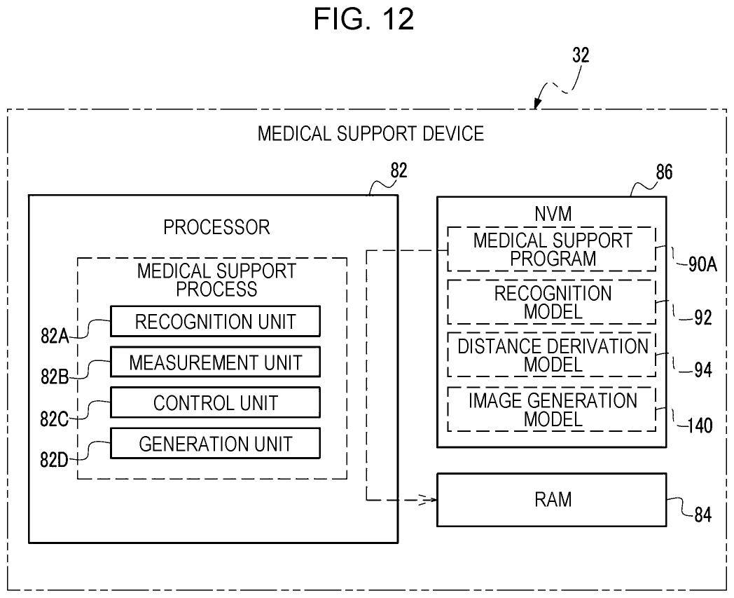

As an example, as illustrated in FIG. 12, the NVM 86 stores a medical support program 90A. The medical support program 90A is an example of a “program” according to the technology of the present disclosure. The processor 82 reads the medical support program 90A from the NVM 86 and executes the read medical support program 90A on the RAM 84. A medical support process according to the second embodiment is implemented by the processor 82 operating as the recognition unit 82A, the measurement unit 82B, the control unit 82C, and a generation unit 82D in accordance with the medical support program 90A executed on the RAM 84.

The NVM 86 stores an image generation model 140. As described in detail below, the image generation model 140 is used by the generation unit 82D.

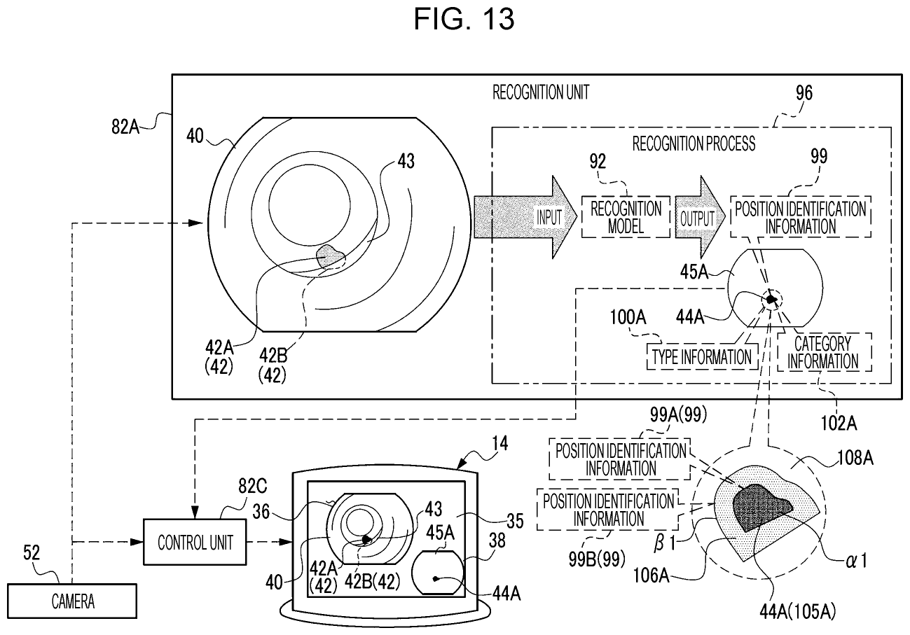

As an example, as illustrated in FIG. 13, a lesion 42 appears in the endoscopic image 40, and a portion of the lesion 42 is blocked by a fold 43. In other words, when the lesion 42 is observed from the position of the camera 52, a portion of the lesion 42 overlaps the fold 43, which is a peripheral region of the lesion 42.

In the second embodiment, the fold 43 is an example of a “peripheral region” according to the technology of the present disclosure. In the second embodiment, the overlap between the lesion 42 and the fold 43 when the lesion 42 is observed from the position of the camera 52 is an example of a “characteristic” and an “overlap between the observation target region and the peripheral region” according to the technology of the present disclosure.

In the example illustrated in FIG. 13, the lesion 42 appearing in the endoscopic image 40 is roughly divided into a visible portion 42A and a non-visible portion 42B. The visible portion 42A is a portion of the lesion 42 appearing in the endoscopic image 40 when the lesion 42 is observed from the position of the camera 52, the portion being a portion not blocked by the fold 43 (that is, a portion not overlapping the fold 43), and is visually recognizable to the doctor 16 through the endoscopic image 40.

By contrast, the non-visible portion 42B is a portion of the lesion 42 appearing in the endoscopic image 40 when the lesion 42 is observed from the position of the camera 52, the portion being a portion blocked by the fold 43 (that is, a portion overlapping the fold 43), and is not visually recognizable to the doctor 16 through the endoscopic image 40.

The recognition unit 82A performs the recognition process 96 on the endoscopic image 40 in which the visible portion 42A appears, in a manner similar to that in the first embodiment described above, to acquire position identification information 99, type information 100A, and category information 102A for the visible portion 42A. The position identification information 99 corresponds to the position identification information 98 described in the first embodiment described above, the type information 100A corresponds to the type information 100 described in the first embodiment described above, and the category information 102A corresponds to the category information 102 described in the first embodiment described above.

The position identification information 99 is roughly divided into position identification information 99A and position identification information 99B. The position identification information 99A corresponds to the position identification information 98A described in the first embodiment described above, and the position identification information 99B corresponds to the position identification information 98B described in the first embodiment described above.

The recognition unit 82A acquires a probability map 45A for the visible portion 42A from the recognition model 92 in a manner similar to that in the first embodiment described above. The probability map 45A corresponds to the probability map 45 described in the first embodiment described above. The probability map 45A includes a segmentation image 44A corresponding to the visible portion 42A. The segmentation image 44A corresponds to the segmentation image 44 described in the first embodiment described above.

In the probability map 45A, the position where the visible portion 42A is present is segmented according to probabilities in a manner similar to that in the first embodiment described above. In the example illustrated in FIG. 13, the probability map 45A is divided into three closed regions, namely, a first segment region 105A, a second segment region 106A, and a third segment region 108A, in accordance with threshold values α1 and β1. The threshold values α1 and β1 correspond to the threshold values α and β described in the first embodiment described above, the first segment region 105A corresponds to the first segment region 105 described in the first embodiment described above, the second segment region 106A corresponds to the second segment region 106 described in the first embodiment described above, and the third segment region 108A corresponds to the third segment region 108 described in the first embodiment described above. As in the first embodiment described above, the segmentation image 44A is formed from the first segment region 105A.

As in the first embodiment described above, the control unit 82C displays the endoscopic image 40 in the first display region 36, and displays the probability map 45A in the second display region 38.

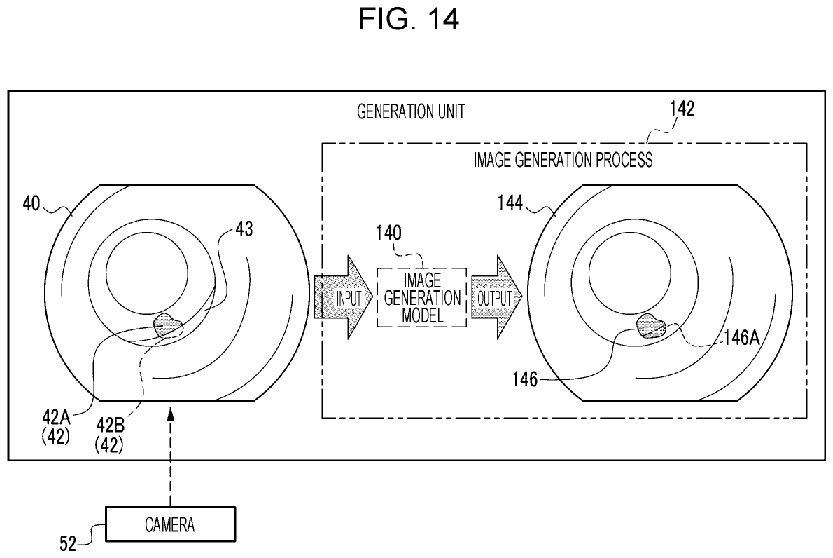

As an example, as illustrated in FIG. 14, the generation unit 82D performs an image generation process 142 on the endoscopic image 40 acquired from the camera 52 (as an example, the endoscopic image 40 on which the recognition process 96 has been performed). The image generation model 140 is used in the image generation process 142. The image generation model 140 is a generation model using a neural network, and is a trained model obtained by training the neural network through machine learning using second training data. An example of the image generation model 140 is an autoencoder such as GANs or VAE.

The second training data used for machine learning to train the neural network in order to generate the image generation model 140 is a dataset including a plurality of pieces of data (that is, data for a plurality of frames) in which second example data and second ground-truth data are associated with each other. The second example data is an image corresponding to the endoscopic image 40 in which a lesion that partially overlaps a peripheral region (for example, a fold, an artificial treatment tool, an organ, and/or the like) appears. The second ground-truth data is an image corresponding to the endoscopic image 40 in which the lesion appears without overlapping the peripheral region.

In the example illustrated in FIG. 14, the generation unit 82D inputs the endoscopic image 40 acquired from the camera 52 to the image generation model 140. In response to this, the image generation model 140 generates a pseudo-image 144 simulating the endoscopic image 40, based on the input endoscopic image 40. The pseudo-image 144 is an image obtained by adding, to the visible portion 42A, a predicted hidden image 146A corresponding to the non-visible portion 42B that overlaps the fold 43 and thus is not visually recognizable from the endoscopic image 40. The image generation model 140 predicts the non-visible portion 42B based on the input endoscopic image 40 to generate the predicted hidden image 146A indicating the prediction result, and combines the predicted hidden image 146A with the visible portion 42A to generate a predicted lesion image 146, thus generating the pseudo-image 144 including the predicted lesion image 146.

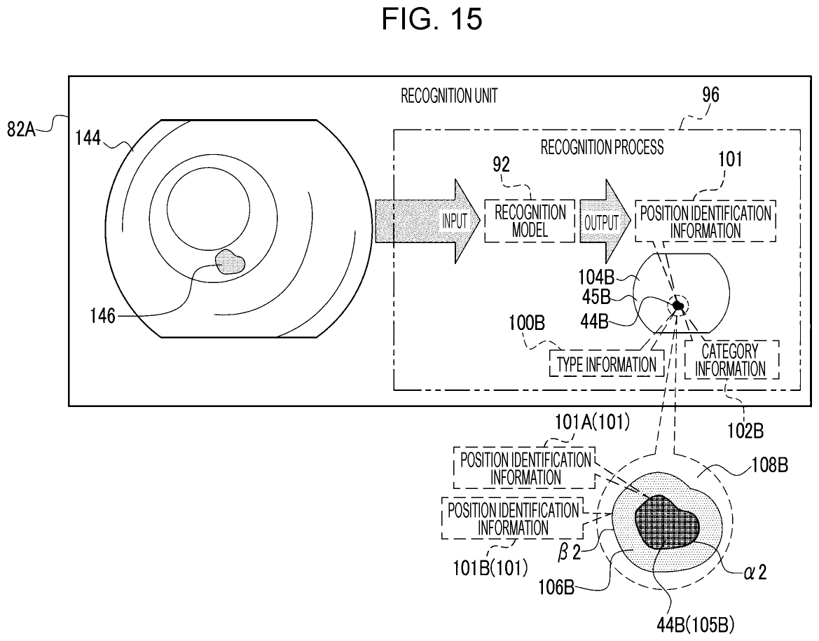

When the pseudo-image 144 is obtained in the way described above, as an example, as illustrated in FIG. 15, the recognition unit 82A performs the recognition process 96 on the pseudo-image 144 including the predicted lesion image 146 in a manner similar to that in the first embodiment described above to acquire position identification information 101, type information 100B, and category information 102B for the predicted lesion image 146. The position identification information 101 corresponds to the position identification information 98 described in the first embodiment described above, the type information 100B corresponds to the type information 100 described in the first embodiment described above, and the category information 102B corresponds to the category information 102 described in the first embodiment described above.

The position identification information 101 is roughly divided into position identification information 101A and position identification information 101B. The position identification information 101A corresponds to the position identification information 98A described in the first embodiment described above, and the position identification information 101B corresponds to the position identification information 98B described in the first embodiment described above.

The recognition unit 82A acquires a probability map 45B for the predicted lesion image 146 from the recognition model 92 in a manner similar to that in the first embodiment described above. The probability map 45B corresponds to the probability map 45 described in the first embodiment described above. The probability map 45B includes a segmentation image 44B corresponding to the predicted lesion image 146. The segmentation image 44B corresponds to the segmentation image 44 described in the first embodiment described above.

In the probability map 45B, the position where the predicted lesion image 146 is present is segmented according to probabilities in a manner similar to that in the first embodiment described above. In the example illustrated in FIG. 15, the probability map 45B is divided into three closed regions, namely, a first segment region 105B, a second segment region 106B, and a third segment region 108B, in accordance with threshold values α2 and β2. The threshold values α2 and β2 correspond to the threshold values a and B described in the first embodiment described above, the first segment region 105B corresponds to the first segment region 105 described in the first embodiment described above, the second segment region 106B corresponds to the second segment region 106 described in the first embodiment described above, and the third segment region 108B corresponds to the third segment region 108 described in the first embodiment described above. As in the first embodiment described above, the segmentation image 44B is formed from the first segment region 105B.

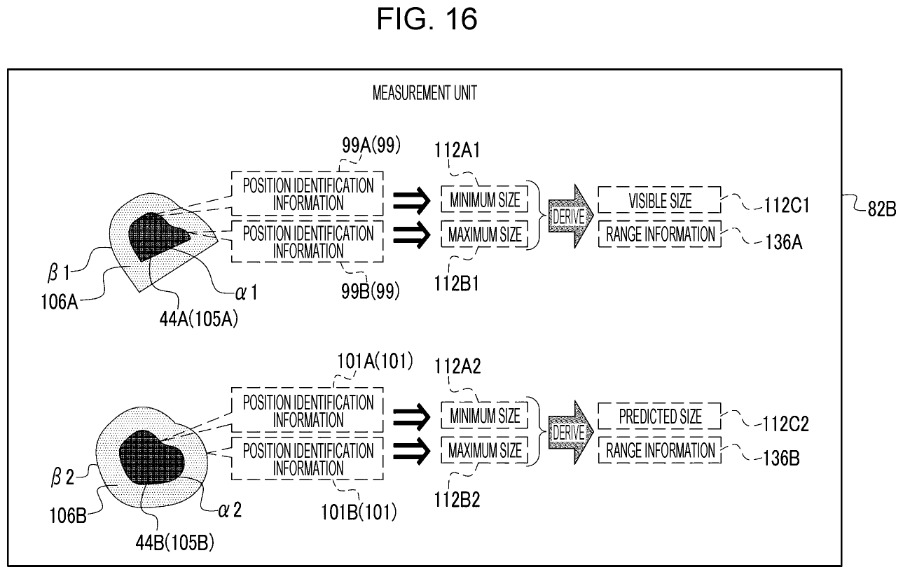

As an example, as illustrated in FIG. 16, the measurement unit 82B derives a visible size 112C1, a predicted size 112C2, range information 136A, and range information 136B in a manner similar to that in the first embodiment described above.

The visible size 112C1 is the size of the visible portion 42A in real space. The visible size 112C1 is derived based on a minimum size 112A1 and a maximum size 112B1. The minimum size 112A1 corresponds to the minimum size 112A described in the first embodiment described above, and is derived based on the position identification information 99A. The maximum size 112B1 corresponds to the maximum size 112B described in the first embodiment described above, and is derived based on the position identification information 99B.

The range information 136A corresponds to the range information 136 described in the first embodiment described above, and is derived based on the minimum size 112A1 and the maximum size 112B1 in a manner similar to that in the first embodiment described above.

The predicted size 112C2 is the size of the predicted lesion image 146 in real space (that is, the size expected to be the actual size of the lesion 42). The predicted size 112C2 is derived based on a minimum size 112A2 and a maximum size 112B2. The minimum size 112A2 corresponds to the minimum size 112A described in the first embodiment described above, and is derived based on the position identification information 101A. The maximum size 112B2 corresponds to the maximum size 112B described in the first embodiment described above, and is derived based on the position identification information 101B.

The range information 136B corresponds to the range information 136 described in the first embodiment described above, and is derived based on the minimum size 112A2 and the maximum size 112B2 in a manner similar to that in the first embodiment described above.

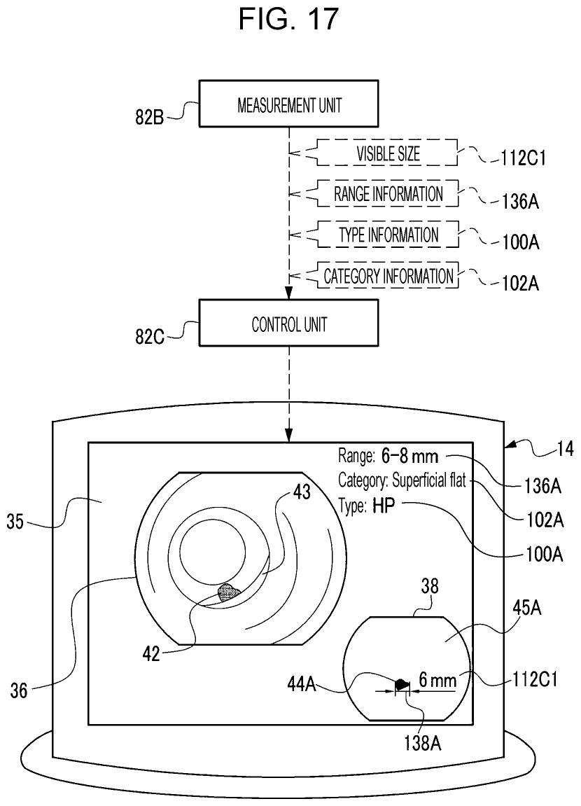

As an example, as illustrated in FIG. 17, the control unit 82C displays the probability map 45A in the second display region 38, and displays the visible size 112C1 on the probability map 45A. The control unit 82C further displays the type information 100A, category information 102A, and the range information 136A on the screen 35.

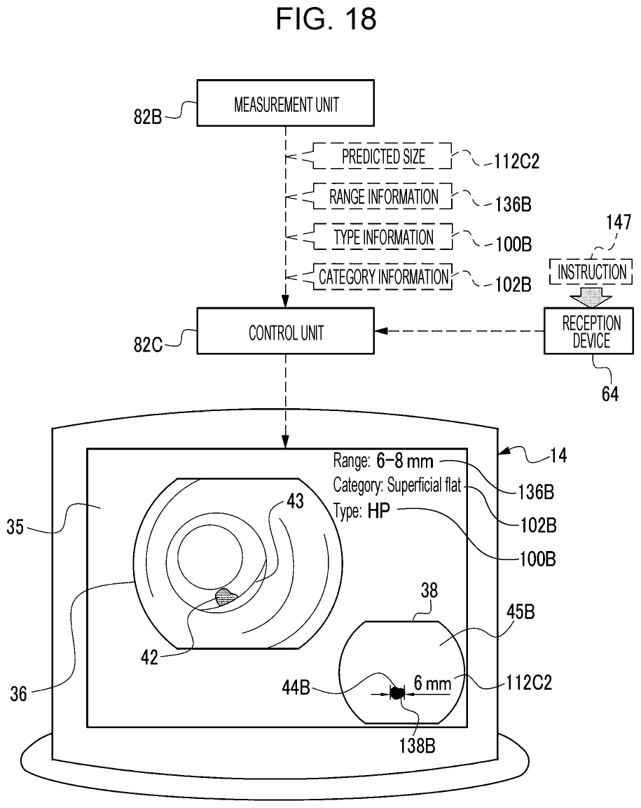

In this state, as an example, as illustrated in FIG. 18, when an instruction 147 to switch the content displayed on the screen 35 (for example, an instruction given from the doctor 16) is received by the reception device 64, the control unit 82C switches the probability map 45A displayed in the second display region 38 to the probability map 45B, and displays the predicted size 112C2 on the probability map 45B. The control unit 82C further switches the type information 100A, the category information 102A, and the range information 136A displayed on the screen 35 to the type information 100B, the category information 102B, and the range information 136B.

In the second embodiment, as described above, the visible size 112C1 and the predicted size 112C2 are displayed on the screen 35. This allows the doctor 16 to grasp the actual size of the lesion 42 in a case where the overlap between the lesion 42 and the fold 43 is included (that is, the size of the visible portion 42A in real space) and the actual size of the lesion 42 in a case where the overlap between the lesion 42 and the fold 43 is not included (that is, the size, in real space, of the lesion 42 constituted by the visible portion 42A and the non-visible portion 42B).

While the second embodiment described above provides an example embodiment in which the display is switched in response to the instruction 147 being received by the reception device 64, this is merely an example. In response to satisfaction of a designated condition (for example, a condition in which a predetermined period of time (for example, 10 seconds) has elapsed since the visible size 112C1 was displayed), the displayed content illustrated in FIG. 17 may be switched to the displayed content illustrated in FIG. 18. Alternatively, the displayed content illustrated in FIG. 17 and the displayed content illustrated in FIG. 18 may be displayed in parallel on one or more screens. In this case, for example, information corresponding to the displayed content illustrated in FIG. 17 and information corresponding to the displayed content illustrated in FIG. 18 may be displayed or hidden in accordance with an instruction received by the reception device 64 and/or various conditions.

### Other Embodiments

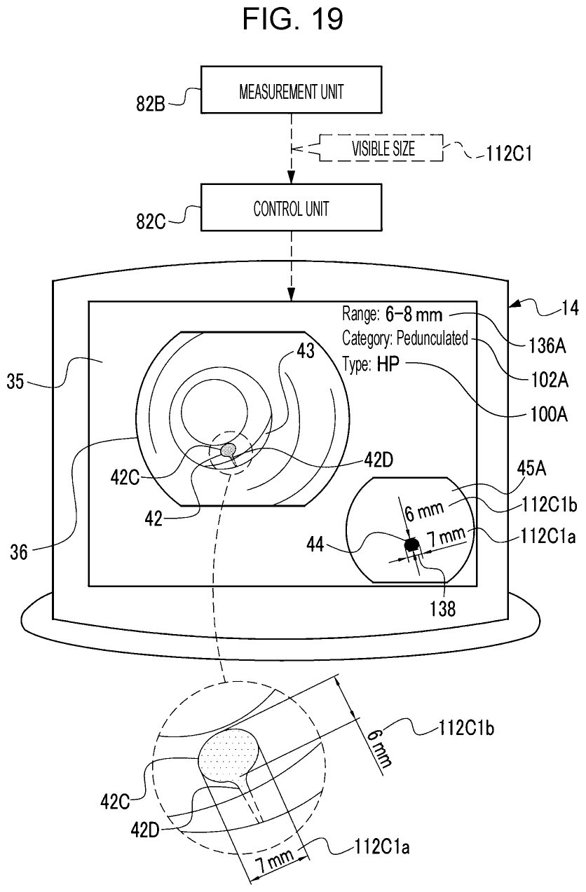

While the superficial flat lesion is presented as an example of the category of the lesion 42 in each of the embodiments described above, this is merely an example. For example, as illustrated in FIG. 19, the technology of the present disclosure is also applicable when the category of the lesion 42 is a pedunculated lesion. In a case where the lesion 42 is pedunculated, the lesion 42 is divided into a tip part 42C and a stem part 42D. It is sufficient that the size of the tip part 42C be measured as the visible size 112C1 and displayed on the screen 35. Alternatively, a visible size 112C1a of a long side of the tip part 42C and a visible size 112C1b of a short side of the tip part 42C may be measured and displayed on the screen 35. In this case, it is desirable to also display the dimension line 138 or the like on the screen 35 to allow the determination of which portion the displayed sizes correspond to.

Alternatively, the visible size 112C1a and the visible size 112C1b may be selectively displayed in accordance with an instruction received by the reception device 64 and/or various conditions. As described above, when sizes in a plurality of directions are measured, it is desirable that the measured sizes in the plurality of directions be selectively displayed on the screen 35 in accordance with an instruction received by the reception device 64 and/or various conditions. The plurality of directions may be determined in accordance with an instruction received by the reception device 64 or may be determined in accordance with various conditions.

While the first embodiment described above provides an example embodiment in which the dimension line 138 is displayed in association with the segmentation image 44 as information that can identify the lesion 42 corresponding to the size 112C displayed on the probability map 45, the technology of the present disclosure is not limited to this. For example, a circumscribed rectangular frame of the segmentation image 44 that can identify the position of the lesion 42 corresponding to the size 112C displayed on the probability map 45 in the endoscopic image 40 may be displayed on the probability map 45. Also in this case, the dimension line 138 may be displayed on the probability map 45 together with the circumscribed rectangular frame. For example, when the position of the lesion 42 is recognized by using AI in a bounding box method, a bounding box may be used as the circumscribed rectangular frame. The same applies to the second embodiment described above.

While the first embodiment described above provides an example embodiment in which the lesion 42 appearing in the endoscopic image 40 is identified from the size and position of the segmentation image 44 on the probability map 45, the technology of the present disclosure is not limited to this. For example, the segmentation image 44 may be superimposed and displayed on the endoscopic image 40. In this case, for example, it is sufficient that the segmentation image 44 be superimposed and displayed on the endoscopic image 40 by using alpha blending. Alternatively, the outer contour of the segmentation image 44 may be superimposed and displayed on the endoscopic image 40. Also in this case, it is sufficient that the outer contour of the segmentation image 44 be superimposed and displayed on the endoscopic image 40 by using alpha blending.

While the first embodiment described above provides an example embodiment in which the minimum size 112A is calculated using the line segment 120 defined by the circumscribed rectangular frame 122, the technology of the present disclosure is not limited to this. The line segment 120 may be set in accordance with an instruction received by the reception device 64. The same applies to the line segment 130 used to calculate the maximum size 112B. Thus, the size 112C in the range designated by the doctor 16 is measured and displayed on the screen 35. The same applies to the second embodiment described above.

While the first embodiment described above provides an example embodiment in which the range information 136 is displayed in text format on the screen 35, this is merely an example. For example, the second segment region 106 may be displayed on the probability map 45 in a display format distinguishable from the segmentation image 44 to allow the doctor 16 to visually recognize the range information 136. Alternatively, the second segment region 106 may be displayed on the endoscopic image 40. Also in this case, it is sufficient that the second segment region 106 be displayed in a display format distinguishable from the lesion 42 appearing in the endoscopic image 40. In this case, for example, it is sufficient that the second segment region 106 be superimposed and displayed on the endoscopic image 40 by using alpha blending. The same applies to the second embodiment described above.

While the first embodiment described above provides an example embodiment in which the size 112C is displayed in the second display region 38, this is merely an example. The size 112C may be displayed outside the second display region 38 in a pop-up manner from within the second display region 38, or the size 112C may be displayed in a region other than the second display region 38 on the screen 35. Various kinds of information such as the category of the lesion 42, the type of the lesion 42, and the width of the lesion 42 may also be displayed in the first display region 36 and/or the second display region 38, or may be displayed on a screen other than the screen 35. The same applies to the second embodiment described above.

Each of the embodiments described above provides an example embodiment in which the size of one lesion 42 is measured and the measurement result is presented to the doctor 16. When a plurality of lesions 42 appear in the endoscopic image 40, it is sufficient that the medical support process be executed on each of the plurality of lesions 42. In this case, a mark or the like may be added to an image region of a lesion 42 corresponding to information (size, category, type, and range) displayed on the screen 35 to allow the identification of which of the lesions 42 the information displayed on the screen 35 corresponds to.

While the first embodiment described above provides an example embodiment in which the size 112C is measured on a frame-by-frame basis, this is merely an example. The statistical value (for example, mean value, median value, mode value, or the like) of the size 112C measured for endoscopic images 40 of a plurality of frames along a time series may be displayed in a display format similar to that in the first embodiment described above. For example, the size 112C may be measured when the amount of shift in the position of the lesion 42 between a plurality of frames is less than a threshold value, and the measured size 112C itself or the statistical value of the size 112C measured for endoscopic images 40 of a plurality of frames along a time series may be displayed on the screen 35. The same applies to the second embodiment described above.

While each of the embodiments described above describes an example embodiment in which the position of the lesion 42 is recognized for each endoscopic image 40 by using an AI-based segmentation method, the technology of the present disclosure is not limited to this. For example, the position of the lesion 42 may be recognized for each endoscopic image 40 by using an AI-based bounding box method.

In this case, it is sufficient that the amount of change of the bounding box be calculated by the processor 82 and it is determined whether to measure the size of the lesion 42, based on the amount of change of the bounding box in a manner similar to that in the embodiment described above.

The amount of change of the bounding box means, for example, the amount of change in the position of the lesion 42. The amount of change in the position of the lesion 42 may be the amount of change in the position of the lesion 42 between adjacent endoscopic images 40 along the time series, or the amount of change in the position of the lesion 42 between three or more frames of endoscopic images 40 along the time series (for example, the statistical value such as the mean value, the median value, the mode value, or the maximum value of the amounts of change between three or more frames of endoscopic images 40 along the time series). Alternatively, the amount of change in the position of the lesion 42 may be the amount of change in the position of the lesion 42 between a plurality of frames along a time series with an interval of one or more frames therebetween.

In each of the embodiments described above, the AI-based object recognition process is presented as an example of the recognition process 96. However, the technology of the present disclosure is not limited to this. The recognition unit 82A may recognize the lesion 42 appearing in the endoscopic image 40 in response to the execution of a non-AI-based object recognition process (for example, template matching or the like).

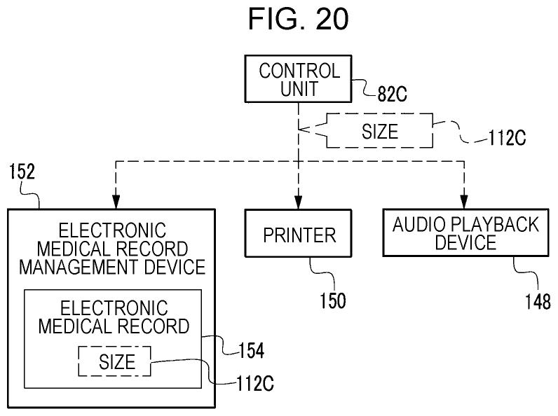

In the first embodiment described above, the size 112C is output to the display device 14, by way of example. However, the technology of the present disclosure is not limited to this, and the size 112C may be output to a destination other than the display device 14. As an example, as illustrated in FIG. 20, the size 112C may be output to an audio playback device 148, a printer 150, an electronic medical record management device 152, and/or the like as a destination.

The size 112C may be output as audio by the audio playback device 148. The size 112C may be printed as a text or the like on a medium (for example, a sheet) or the like by the printer 150. The size 112C may be stored in an electronic medical record 154 managed by the electronic medical record management device 152. The same applies to the second embodiment described above.

While the first embodiment described above describes an example embodiment in which the arithmetic expressions 124, 134, and 135 are used to calculate the size 112C, the technology of the present disclosure is not limited to this. The size 112C may be measured by performing an AI-based process on the endoscopic image 40. In this case, for example, a trained model is used that, in response to an input of the endoscopic image 40 including the lesion 42, outputs the size 112C of the lesion 42. To generate the trained model, deep learning is performed on a neural network by using training data in which lesions appearing in images used as example data are assigned annotations indicating the sizes of the lesions as ground-truth data. The same applies to the second embodiment described above.

While each of the embodiments described above provides an example embodiment in which the recognition unit 82A performs the recognition process 96 on the endoscopic image 40 acquired from the camera 52 to recognize the characteristics of the lesion 42 appearing in the endoscopic image 40, the technology of the present disclosure is not limited to this. For example, the characteristics of the lesion 42 appearing in the endoscopic image 40 may be provided to the processor 82 from the doctor 16 or the like through the reception device 64 or the like, or may be acquired by the processor 82 from an external device (for example, a server, a personal computer, a tablet terminal, and/or the like). Also in these cases, it is sufficient that the measurement unit 82B measure a size corresponding to the characteristics of the lesion 42 in a manner similar to that in the embodiments described above.

While each of the embodiments described above describes an example embodiment in which the distance information 114 is derived using the distance derivation model 94, the technology of the present disclosure is not limited to this. Other methods for deriving the distance information 114 using an AI-based method include, for example, a method for combining segmentation and depth estimation (for example, regression learning to provide the distance information 114 to the entire image (for example, all the pixels constituting the image) or unsupervised learning to learn the distance of the entire image in an unsupervised way).

While each of the embodiments described above provides an example embodiment in which the distance from the camera 52 to the intestinal wall 24 is derived by using an AI-based method, the distance from the camera 52 to the intestinal wall 24 may be actually measured. In this case, for example, the tip portion 50 (see FIG. 2) may be provided with a distance-measuring sensor, and the distance from the camera 52 to the intestinal wall 24 may be measured by the distance-measuring sensor.

While the endoscopic image 40 is illustrated in each of the embodiments described above, the technology of the present disclosure is not limited to this. The technology of the present disclosure is also applicable to a medical image (for example, an image obtained by a modality other than the endoscope 12, such as a radiographic image or an ultrasound image) other than the endoscopic image 40.

While each of the embodiments described above provides an example embodiment in which the size 112C of the lesion 42 appearing in a moving image is measured, this is merely an example. The technology of the present disclosure is also applicable to a stop-motion image or a still image in which the lesion 42 appears.

While each of the embodiments described above provides an example embodiment in which the distance information 114 extracted from the distance image 116 is input to the arithmetic expressions 124 and 134, the technology of the present disclosure is not limited to this. For example, it is sufficient that the distance information 114 corresponding to the position identified from the position identification information 98 be extracted from among all the pieces of distance information 114 output from the distance derivation model 94, without the generation of the distance image 116, and the extracted distance information 114 be input to the arithmetic expressions 124 and 134.

While each of the embodiments described above provides an example embodiment in which the medical support process is performed by the processor 82 of the computer 78 included in the endoscope 12, the technology of the present disclosure is not limited to this, and a device external to the endoscope 12 may perform the medical support process. Examples of the device external to the endoscope 12 include at least one server and/or at least one personal computer connected to the endoscope 12 in a communicable manner. Alternatively, the medical support process may be performed by a plurality of devices in a distributed manner.

While each of the embodiments described above provides an example embodiment in which the medical support programs 90 and 90A (hereinafter referred to as “medical support programs” without reference numerals) are stored in the NVM 86, the technology of the present disclosure is not limited to this. For example, the medical support programs may be stored in a portable non-transitory computer-readable storage medium such as an SSD or a USB memory. The medical support programs stored in the non-transitory storage medium are installed in the computer 78 of the endoscope 12. The processor 82 executes the medical support process in accordance with the medical support programs.

Alternatively, the medical support programs may be stored in a storage device of another computer, a server, or the like connected to the endoscope 12 via a network, and the medical support programs may be downloaded in response to a request from the endoscope 12 and installed in the computer 78.

Not all, but a portion, of the medical support programs may be stored in a storage device of another computer, a server device, or the like connected to the endoscope 12, or not all, but a portion, of the medical support programs may be stored in the NVM 86.

Examples of a hardware resource that executes the medical support process may include the following various processors. The processors include, for example, a CPU that is a general-purpose processor configured to execute software, that is, a program, to function as a hardware resource that executes the medical support process. The processors further include, for example, a dedicated electric circuit that is a processor having a circuit configuration designed specifically for executing specific processing, such as an FPGA, a PLD, or an ASIC. Each of the processors incorporates or is connected to a memory, and uses the memory to execute the medical support process.

The hardware resource that executes the medical support process may be configured as one of the various processors or as a combination of two or more processors of the same type or different types (for example, a combination of a plurality of FPGAs or a combination of a CPU and an FPGA). The hardware resource that executes the medical support process may be a single processor.

Examples of configuring the hardware resource as a single processor include, first, a form in which a single processor is configured as a combination of one or more CPUs and software and the processor functions as a hardware resource that executes the medical support process. The examples include, second, a form in which, as typified by an SoC or the like, a processor is used in which the functions of the entire system including a plurality of hardware resources that execute the medical support process are implemented as one IC chip. As described above, the medical support process is implemented by using one or more of the various processors described above as hardware resources.

More specifically, the hardware structure of these various processors may be an electric circuit in which circuit elements such as semiconductor elements are combined. The medical support process described above is merely an example. Thus, it goes without saying that unnecessary steps may be deleted, new steps may be added, or the processing order may be changed without departing from the gist.

The description and drawings presented above provide detailed descriptions of portions according to the technology of the present disclosure and are merely examples of the technology of the present disclosure. For example, the descriptions related to the configurations, functions, operations, and effects described above are descriptions related to an example of the configurations, functions, operations, and effects of portions according to the technology of the present disclosure. Thus, it goes without saying that unnecessary portions may be deleted or new elements may be added or substituted in the description and drawings presented above without departing from the gist of the technology of the present disclosure. To avoid complexity and facilitate understanding of portions according to the technology of the present disclosure, descriptions related to common general technical knowledge and the like, for which no specific explanation is required to implement the technology of the present disclosure, are omitted in the description and drawings presented above.

As used herein, “A and/or B” is synonymous with “at least one of A or B”. That is, “A and/or B” means only A, only B, or a combination of A and B. In this specification, furthermore, a concept similar to that of “A and/or B” is applied also to the expression of three or more matters in combination with “and/or”.

All publications, patent applications, and technical standards described herein are incorporated herein by reference to the same extent as if each individual publication, patent application, and technical standard were specifically and individually indicated to be incorporated by reference.

What is claimed is:

1. A medical support device comprising:
a processor,
the processor being configured to:
recognize, using a medical image, an observation target region appearing in the medical image;
measure a size corresponding to a characteristic of the observation target region, based on the medical image; and
output the size.

2. The medical support device according to claim 1, wherein
the characteristic includes a shape of the observation target region, a category of the observation target region, a type of the observation target region, clarity of a contour of the observation target region, and/or an overlap between the observation target region and a peripheral region.

3. The medical support device according to claim 1, wherein
the processor is configured to recognize the characteristic, based on the medical image.

4. The medical support device according to claim 1, wherein
the size is a long side, a short side, a radius, and/or a diameter of the observation target region.

5. The medical support device according to claim 1, wherein
the observation target region is recognized by a method using AI, and
the size is measured based on a probability map obtained from the AI.

6. The medical support device according to claim 5, wherein
the size is measured based on a closed region obtained by dividing the probability map according to a threshold value.

7. The medical support device according to claim 5, wherein
the size is measured based on a plurality of segment regions obtained by dividing the probability map according to a plurality of threshold values.

8. The medical support device according to claim 7, wherein
the size has a range, and
the range is identified based on the plurality of segment regions.

9. The medical support device according to claim 8, wherein
a lower limit value of the range is measured based on a first segment region that is smallest among the plurality of segment regions, and
an upper limit value of the range is measured based on a second segment region that is an outer region with respect to the first segment region among the plurality of segment regions.

10. The medical support device according to claim 1, wherein
the processor is configured to measure a plurality of first sizes of the observation target region, based on the medical image, and
the size is a representative value of the plurality of first sizes.

11. The medical support device according to claim 10, wherein
the representative value includes a maximum value, a minimum value, a mean value, a median value, and/or a variance value.

12. The medical support device according to claim 1, wherein
the characteristic includes an overlap between the observation target region and a peripheral region, and
the size is a size of the observation target region in a case where the overlap is included and/or a size of the observation target region in a case where the overlap is not included.

13. The medical support device according to claim 1, wherein
the size is output by displaying the size on a screen.

14. The medical support device according to claim 1, wherein
the medical image is an endoscopic image obtained by imaging with an endoscope.

15. The medical support device according to claim 1, wherein
the observation target region is a lesion.

16. An endoscope comprising:
the medical support device according to any claim 1; and
a module to be inserted into a body including the observation target region to acquire the medical image by imaging the observation target region.

17. A medical support method comprising:
recognizing, using a medical image, an observation target region appearing in the medical image;
measuring a size corresponding to a characteristic of the observation target region, based on the medical image; and
outputting the size.

18. A non-transitory computer-readable storage medium storing a program executable by a computer to execute a medical support process,
the medical support process comprising:
recognizing, using a medical image, an observation target region appearing in the medical image;
measuring a size corresponding to a characteristic of the observation target region, based on the medical image; and
outputting the size.
